(19) United States
(12) Patent Application Publication
(10) Pub. No.: US 2013/0034499 A1
(43) Pub. Date: Feb. 7, 2013

(54) SGII AS A PROGNOSTIC MARKER IN CONDITIONS WHICH REQUIRE CRITICAL CARE

(71) Applicants: Helge Røsjø, Oslo (NO); Geir Christensen, Oslo (NO); Torbjørn Omland, Oslo (NO); Mats Stridsberg, Uppsala (SE)

(72) Inventors: Helge Røsjø, Oslo (NO); Geir Christensen, Oslo (NO); Torbjørn Omland, Oslo (NO); Mats Stridsberg, Uppsala (SE)

(73) Assignee: Universitetet I Oslo, Oslo (NO)

(21) Appl. No.: 13/508,798

(22) PCT Filed: Nov. 15, 2010
(86) PCT No.: PCT/GB2010/002105

§ 371 Date: Aug. 23, 2012

(30) Foreign Application Priority Data
Nov. 13, 2009 (GB) 0919901.9

(51) Int. Cl. G01N 33/566; A61K 49/00; C07K 16/18; G01N 21/64

(52) U.S. Cl. 424/9.2; 436/501; 435/7.92; 435/7.94; 435/7.93; 424/9.1; 530/389.8

(57) ABSTRACT

The present invention relates to methods for determining the prognosis of a condition which requires critical care, said methods comprising determining the level of SgII, or fragments thereof in a subject. The methods of the invention can also be used to determine whether an individual is suffering from a condition which requires critical care and determining the severity of a condition which requires critical care.

The present invention relates to methods for diagnosing or determining severity of a condition which requires critical care in a subject (i.e. methods for diagnosing or determining severity of critical illness). The invention in particular relates to establishing prognosis for such subjects. The methods are particularly useful for diagnosing or determining severity and prognosis of conditions which require admittance to intensive care units (ICUs) or similar settings. The methods also relate to diagnosis of specific pathophysiological pathways activated in patients requiring critical care, and for use in monitoring of patients requiring critical care. The invention is especially useful for diagnosing circulatory failure (medical shock) in patients with critical illness, particularly sepsis.

The ability to determine severity of illness and to prognose patient outcome is of significant importance to physicians caring for patients requiring critical care, i.e. critically ill patients.

Several severity scoring systems have been developed for use in intensive care unit (ICU) management, such as SAPS (Simplified Acute Physiology Score) II, SOFA (Sequential Organ Failure Assessment), APACHE (Acute Physiology and Chronic Heath Evaluation) and LOD (Logistic Organ Dysfunction).

However, such systems suffer from disadvantages. For example, they are very complex in that they require the analysis of a large number of parameters (17 for SAPS II and 6 for SOFA). In addition, the scores can generally only be calculated after 24 hours of hospitalization.

Thus, the identification of a single biomarker which could be measured quickly and easily upon the presentation of such patients to a physician or upon admission of a patient to a critical care setting and would provide information regarding severity of condition and prognosis would be extremely advantageous. Such a biomarker could be used alone or in combination with other predictors of severity, such as the scoring systems described above. For complementary use the candidate biomarker should provide incremental information to the information obtained from patient history, clinical evaluation, additional diagnostic and prognostic tools such as standard biochemical analysis and established scoring systems. The potential of candidate biomarkers may be evaluated by statistical analysis such as the Student' t test or the Mann-Whitney U test, correlation analysis (Pearson or Spearman), multivariate logistic or Cox proportional hazard analysis, and the use of receiver operating characteristics analysis (ROC-AUC), or the more recently implemented methods of re-calibration of a risk model (Rosjo H, et al. Clin Sci 2009; 117:13-15). Preferable biomarkers with potential to improve accuracy will be only moderately or poorly correlated with established markers of risk, and will be independently associated with the outcome in multivariable statistical models (Rosjo H, et al. Scand J Clin Lab Invest 2008; 68:673-677). These markers will often be found to represent processes currently not being monitored by traditional diagnostic and prognostic tools, and thus will also be of interest for diagnosis and monitoring of patients. However, strictly speaking, a biomarker does not need to be directly associated with the process, but merely reflective of the process (Røsjø H, et al. Scand J Clin Lab Invest 2008; 68:673-677).

Secretogranin II (SgII, Sg2, prosecretoneurin, or chromogranin C) is a protein of 67 kDa (calculated molecular weight), that is part of a family of acidic proteins called the granin protein family.

The most investigated protein in this family is chromogranin A (CgA). During the past two decades, CgA has been used clinically as a diagnostic biomarker for neuroendocrine tumors, such as pheochromocytomas, carcinoids and neuroblastomas (O'Connor D T et al., N Engl J Med 1986; 314:1145-51, Syversen U et al., Eur J Gastroent Hepatol 1993; 5:1043-1050, Hsiao R J et al., J Clin Invest 1990; 85:1555-1559). CgA level has also been shown to increase with severity of heart failure (Ceconi C et al., Eur Heart J 2002; 23:967-974), and to be an independent predictor of mortality and heart failure development in different cohorts of patients with acute coronary syndromes (Omland T et al., Am J Med 2003; 14:25-30, Estensen M E et al., Am Heart J. 2006; 152:927.e1-e6, Jansson A M et al., Eur Heart J. 2009; 30:25-32).

A confounding factor however, possibly reducing CgA's merit as a biomarker, is the increase in CgA levels seen in various diseases and treatments. Examples of diseases known to elevate the blood concentration of CgA include pheochromocytomas, carcinoid tumors, neuroblastomas, neuroendocrine tumors, neurodegenerative diseases, chronic heart failure, acute myocardial infarction, chronic renal failure, hepatic failure, ongoing steroids treatment, or ongoing proton pump inhibitors treatment, or ongoing histamine receptor type 2 blockers (H2-blockers) treatment or surgical intervention. Thus, any patient found to have an increase in CgA levels may equally be suffering from one of these diseases or undergoing one of these treatments (or indeed another disease or treatment which elevates the level of CgA), as opposed to the disease for which CgA is being used as a biomarker.

Much less is currently known about CgB and even less is known about SgII (and indeed the other members of the granin protein family). However, both CgB and SgII are known to act as pro-peptides from which other peptides are produced (Taupenot L et al., New Engl J Med 2003; 348:1134-1149, Helle KB. Biol Rev. 2004; 79:769-794).

Studies on CgB and SgII are thus quite minimal at this stage. For example, although the measurement of circulating CgB has been carried out in patients with neuroendocrine tumours (Stridsberg et al., 2007, Reg Peptides, 139:80-83), CgB was shown to be significantly less sensitive as a biomarker for neuroendocrine tumours than CgA (Stridsberg M et al., 2005, Reg Peptides, 125: 193-199; Stridsberg M et al., 2004, Reg Peptides, 117: 219-227). The same is the case for SgII (Stridsberg M et al., 2008, Reg Peptides, 148: 95-98).

Indeed, SgII is known to be present at a much lower level than CgA or CgB in blood (and other body fluids and tissues) of normal individuals (Stridsberg M et al., 2005, Reg Peptides; 125: 193-199, Stridsberg M et al., 2004, Reg Peptides; 117: 219-227 Stridsberg M et al., 2008, Reg Peptides; 148: 95-98). Thus, the rationale for the potential use of circulatory levels, or levels in other body fluids or in tissues, of SgII as a biomarker of disease are uncertain and as yet unproven. In the present invention it has been found that SgII is a useful biomarker to determine prognostic or other information for patients with critical illness in general (i.e. the prognostic or other use does not appear to be restricted to particular conditions underlying the critical illness). In particular, we have found measurement of SgII to be superior to measurement of CgB and CgA for prognostic information in patients with severe sepsis and septic shock, thus indicating that SgII may be a superior biomarker to other markers of the granin family in patients with critical illness. SgII as a biomarker has also been found to be superior to the established biomarkers NT-proBNP and highly sensitive troponin T (hs-cTnT) in critical illness.

Thus, before the present invention, there was no suggestion in the art that levels of SgII, in particular circulating levels of SgII, could be used as a biomarker for determining severity of illness in a subject suffering from a condition which requires critical care, e.g. which requires admittance to an intensive care unit (ICU). Surprisingly however, it has now been found that levels of SgII in a subject, for example in a body fluid of said subject, can be used to diagnose or determine severity of a condition which requires critical care, e.g. a condition which requires admittance to an intensive care unit (ICU), in particular sepsis or septic shock. In addition, levels of SgII in a subject, for example in a body fluid of said subject, can be used in the prognosis of the future severity, course and outcome of critical illness.

SgII has thus been found to be a new biomarker that is related to the severity of illness and prognosis of illness in individual critical care patients.

Importantly and advantageously, the methods of the present invention involving the measurement of levels of SgII to diagnose or to determine severity or prognosis of conditions which require critical care have advantages over prior art methods involving the use of severity scoring systems such as SAPS II and SOFA in that they only require the measurement of one variable and are thus much quicker and less complex. Indeed, the methods can readily and advantageously be carried out upon admission to the critical care setting or at least on the first day of admission or within the first 24 or 48 hours.

Area under the receiver-operating characteristic curve (ROC-AUC) analysis is considered an excellent test for evaluating prognostic utility for a biomarker (Pepe M S et al., 2004, Am. J. Epidemiology; 159: 882-890). When comparing prognostic accuracy for mortality for SgII, SOFA and SAPS II in the same cohort of critically ill patients, similar ROC-AUC values for SgII compared to SOFA and SAPS II are observed. SgII has thus been found to have non-inferior sensitivity and specificity for the prognosis of critically ill patients as the recognised prior art methods, with the additional advantage of being far simpler and quicker to perform (SAPS II and SOFA require the analysis of 17 and 6 parameters, respectively). A further advantage for SgII measurement over prior art methods is less variability, as SgII measurement is not based on parameters which are subject to inter- and intra-observer variability such as found for conventional scoring models: SAPS: heart rate, systolic blood pressure, respiratory rate, body temperature, Glascow Coma Scale; and SOFA: blood pressure, temperature.

Thus, in one aspect the present invention provides a method of diagnosing or determining whether an individual is suffering from a condition which requires critical care, said method comprising determining the level of SgII, or fragments thereof, in a subject.

The methods of the invention can also be used to determine the severity of said critical care condition in said subject. Thus, in a further aspect the present invention provides a method of determining severity of a condition which requires critical care, said method comprising determining the level of SgII, or fragments thereof, in a subject suffering from said condition.

Advantageously, said methods allow a prognosis of said condition to be made. Thus, a yet further aspect of the present invention provides a method for the prognosis of a condition which requires critical care, said method comprising determining the level of SgII, or fragments thereof, in a subject suffering from said condition.

The term “prognosis” as used herein refers to and includes a risk prediction of the severity of disease or of the probable course and clinical outcome associated with a disease. Associated with this is also the ability to classify or discriminate patients according to the probability of whether various treatment options may be of gain or detrimental to an individual, i.e. the use of SgII, or fragments thereof, to guide treatment. In the case of critical care conditions, which are the subject of the present invention, said prediction of course and clinical outcome includes a prediction of any clinically relevant course or outcome, for example predicting morbidity or mortality rate, likelihood of recovery, likelihood of hospital admission, or in general predicting the speed of critical illness development, and the speed of recovery. Preferably the prognostic methods of the present invention are used to predict morbidity or mortality. Thus, the risk of morbidity and mortality is increased in patients with high or increased levels of SgII (or fragments thereof). The methods of the invention can be useful to predict short term prognosis or mortality (e.g. ≦30 days mortality) or long term prognosis or mortality (e.g. >30 days mortality). Alternatively, the prognostic methods of the present invention are used to predict the likelihood of a critically ill patient developing circulatory failure (septic shock).

The terms “condition which requires critical care”, “critical care condition” and “critical illness” are used interchangeably herein to refer to conditions, injuries, diseases, disorders or any other illnesses which are life threatening to the sufferer and may thus result in death with a relatively short period of time (e.g. hours or days). Such conditions require critical care (e.g. monitoring and treatment) that generally involves close, constant attention by a team of specially trained health professionals. Such care usually takes place in an intensive care unit (ICU) or trauma centre. However, care might take place in any appropriate unit which has a similar or equivalent structure and capability as an ICU or trauma centre.

Thus, preferred critical conditions for application of the methods of the present invention are conditions requiring admittance to an ICU or a setting which has a similar or equivalent structure and capability such as a trauma centre and preferred patients are ICU patients or trauma centre patients. Such conditions include complications from surgery, life threatening accidents or other life threatening physical trauma or stress, medical shock (i.e. a condition when insufficient blood flow reaches body tissues), infections (e.g. bacterial, fungal or viral infections), organ dysfunction, single or multi-organ failure (MOF), poisoning and intoxication, severe allergic reactions and anaphylaxis, acute gastrointestinal and abdominal conditions resulting in the Systemic Inflammatory Response Syndrome/SIRS (Levy M M, et al. Crit. Care Med 2003; 31:1250-1256), burn injury, acute cerebral hemorrhage or infarction, and severe respiratory problems. It should be noted that, by their very nature, such conditions which require critical care are serious, severe, life-threatening forms of illness. Thus, for any diseases or conditions referred to herein which also have non life-threatening forms or stages, the invention relates to severe life-threatening forms or stages of the condition, e.g. such forms or stages as require admission to an ICU or similar setting.

Methods of the present invention have been shown to work in sepsis and related conditions such as septic shock. However, the methods have also been shown to work in other patients (i.e. non-septic patients) suffering from critical illness, for example ICU patients without infection, in particular mechanically ventilated ICU patients. Thus, SgII seems to be a powerful new biomarker in critical illness that is not restricted to the particular underlying condition.

Particular examples of conditions include sepsis, severe sepsis (sepsis with organ dysfunction), septic shock (sepsis with acute circulatory failure) (for sepsis-related definitions see Levy M M, et al. Crit. Care Med 2003; 31:1250-1256 and the definitions provided by the American College of Chest Physicians and the Society of Critical Care Medicine, Crit. Care Med. 1992; 20:864-874), Acute Respiratory Distress Syndrome (ARDS) defined by pulmonary and systemic inflammation and pulmonary endothelial and epithelial injury that result in alveolar filling and respiratory failure (Bajwa E K, et al. Crit. Care Med 2007; 35:2484-2490), severe pneumonia, respiratory failure (particularly acute respiratory failure), respiratory distress, severe Chronic Obstructive Pulmonary Disease (COPD), trauma, subarachnoidal haemorrhage (SAH), severe stroke, asphyxia, gastrointestinal disease, intoxication, neurological conditions, any condition for which the patient requires mechanical ventilation and complications from surgery. Preferred examples are sepsis, severe sepsis, septic shock, pneumonia, COPD, SAH and severe stroke, trauma, acute respiratory failure, gastrointestinal disease, intoxication, neurological conditions, any condition for which the patient requires mechanical ventilation and complications from surgery. Especially preferred examples are sepsis, severe sepsis or septic shock.

It is particularly useful to be able to identify critically ill patients with sepsis that will develop circulatory failure (septic shock/medical shock) as these patients are likely to benefit from targeted therapy with specific drugs such as inotropic drugs (e.g. dopamine, dobutamine, norepinephrine, etc) to maintain adequate blood pressure of or sustained perfusion of the peripheral organs and these drugs would not be given to critically ill patients generally.

By way of example, when it comes to definitions of sepsis and related diseases, according to the American College of Chest Physicians and the Society of Critical Care Medicine, the different levels of sepsis are defined as follows:

“Systemic inflammatory response syndrome (SIRS)” is defined by the presence of two or more of the following findings: 1) Body temperature<36° C. (97° F.) or >38° C. (100° F.) (hypothermia or fever), 2) Heart rate>100 beats per minute (tachycardia), 3) Respiratory rate>20 breaths per minute or, on blood gas, a PₐCO₂ less than 32 mm Hg (4.3 kPa) (tachypnea or hypocapnia due to hyperventilation), 4) White blood cell count<4,000 cells/mm³ or >12,000 cells/mm³ (<4×10⁹ or >12×10⁹ cells/L), or greater than 10% band forms (immature white blood cells). (leukopenia, leukocytosis, or bandemia).

“Sepsis” is defined as SIRS in response to a confirmed infectious process. Infection can be suspected or proven (by culture, stain, or polymerase chain reaction (PCR)), or a clinical syndrome pathognomonic for infection. Specific evidence for infection includes WBCs in normally sterile fluid (such as urine or cerebrospinal fluid (CSF), evidence of a perforated viscus (free air on abdominal x-ray or CT scan, signs of acute peritonitis), abnormal chest x-ray (CXR) consistent with pneumonia (with focal opacification), or petechiae, purpura, or purpura fulminans.

“Severe sepsis” is defined as sepsis with organ dysfunction, hypoperfusion, or hypotension.

“Septic shock” is defined as sepsis with refractory arterial hypotension or hypoperfusion abnormalities in spite of adequate fluid resuscitation. Signs of systemic hypoperfusion may be either end-organ dysfunction or serum lactate greater than 4 mmol/dL. Other signs include oliguria and altered mental status. Patients are defined as having septic shock if they have sepsis plus hypotension after aggressive fluid resuscitation (typically upwards of 6 liters or 40 ml/kg of crystalloid).

Whilst not wishing to be bound by theory, the inventors believe that increased SgII levels are generally associated with pathophysiological pathways which become activated in the various stresses and traumas suffered by critically ill patients with life threatening conditions, e.g. patients which require admittance to an ICU (ICU patients). Thus, although examples of specific critical care conditions are mentioned herein, it is believed that the determination of SgII levels as described herein has general applicability in the diagnosis, determination of severity of, and prognosis of critical illness (i.e. conditions which require critical care).

Although critical care will generally be carried out in an ICU or similar setting, this does not preclude the methods of the invention being carried out in a different setting, e.g. a non-hospital setting such as a Doctor's surgery or clinic, in order to identify high risk patients which should then be admitted to an ICU or trauma centre. Indeed, this scenario is contemplated by the present invention. In addition, although the subjects suffering from critical illness will generally have been cared for in an ICU or similar setting at some point in their treatment, the methods of the invention are equally useful to determine the severity or prognose critical care conditions in subjects which remain in the ICU (or similar setting) or in subjects which are moved from the ICU (or similar setting) to another setting, e.g. another in-hospital setting. Thus, when the prognostic end point is mortality, the methods of the invention can be used to determine both in-hospital and ICU mortality.

The methods of the invention, or at least the taking of samples from a subject upon which such methods will be carried out, can be carried out at any appropriate time point after the subject comes into contact with medical personnel or medical professionals or enters or is admitted to a medical institution or critical care setting. However, it is beneficial if the methods are carried out (or the samples are taken) on subjects within 72 hours of contact with a Doctor or other medical personnel or professional, e.g. within 72 hours after an appointment with a Doctor or other medical personnel or professional, or within 72 hours of admission or arrival at a medical institution or critical care setting such as a hospital (e.g. at an emergency ward, or accident and emergency department), clinic, ICU or trauma centre. In preferred embodiments of the invention, the methods are carried out (or the samples taken) on the first day of contact, admission or arrival, or within the first 24 or 48 hours of contact, admission or arrival, e.g. within 48 hours, 24 hours, 18 hours, 12 hours, 6 hours, 4 hours or 2 hours of contact, admission or arrival. Most preferably the methods of the invention are carried out (or the samples taken) on admission or arrival, or during the first contact of a patient with medical personnel or professionals (e.g. during their first appointment with a Doctor), or on the day of admission to hospital or critical care setting such as an ICU or trauma centre, or other medical institution. In preferred embodiments, the methods of the invention are carried out (or samples are taken) in the ICU (or similar setting).

As symptoms and clinical findings may vary greatly between patients with critical illness, determining presence (diagnosis), severity and prognosis of critical illness, and sepsis and septic shock especially, may be difficult. Currently there is no reference method to early establish patients at the highest risk, e.g. to diagnose critical illness per se. At present, diagnosis of critical illness is based mainly on the physicians subjective evaluation of the patient's cardiovascular status, e.g. blood pressure and heart rate, and respiratory and cognitive status, e.g. awake and mentally alert, all subject to large physician inter- and intra-observer variability. The established risk models for ICU patients are of limited use in the early evaluation of patients for critical illness as they are time consuming, not easy to perform and calculate at the patient's bedside, and requires 24 hours for data collection.

Thus, methods such as that of the present invention, which allow for determination via a simple test for a biomarker which can be quickly and easily carried out on a readily obtainable sample such as for example a blood sample or other easily available biological or body fluid (e.g. a. urine or a saliva test), are much in demand.

The methods of the invention may optionally comprise comparing the level of SgII, or fragments thereof, found in said subject to a control level.

It should be noted however that although the control level for comparison would generally be derived by testing an appropriate set of control subjects, the methods of the invention would not necessarily involve carrying out active tests on such a set of control subjects but would generally involve a comparison with a control level which had been determined previously from control subjects.

An increased level of SgII, or fragments thereof, in a subject is indicative of a severe critical care condition and poor prognosis. An increased level of SgII, or fragments thereof, may also be used to diagnose such critical care conditions.

Preferably the level of SgII, or a fragment thereof, is determined by analyzing a test sample which is obtained from or removed from said subject by an appropriate means. The determination is thus preferably carried out in vitro.

For a positive diagnosis to be made or for a condition to be determined as severe or for a poor prognosis to be determined, the level of SgII in the test sample or subject is increased, preferably significantly increased, compared to the level found in an appropriate control sample or subject. More preferably, the significantly increased levels are statistically significant, preferably with a probability value of <0.05. Where the control sample or subjects are healthy control subjects, a typical control level of SgII for comparison is an age and gender specific reference value, for example a value above a pre-specified threshold in a healthy population. Where the control sample or subjects are healthy control subjects, a typical control level of SgII for comparison is approximately 0.12 or 0.13 nmol/L, e.g. less than 0.13, 0.14 or 0.15 nmol/L, or a range of 0.10 nmol/L to 0.15 nmol/L.

Viewed alternatively, an increase in level of the SgII of ≧10%, ≧15%, ≧20%, ≧25%, ≧30% or ≧35% compared to the level found in an appropriate control sample or subject (i.e. when compared to a control level) is indicative of a positive diagnosis, a severe condition or a poor prognosis. On the other hand, if SgII is measured repeatedly during or after hospitalization, a decrease in SgII levels by 10% or more, 15% or more, or 20% or more, compared to a previously measured SgII value, is indicative of the absence of disease or of a less severe condition or a good prognosis.

Alternatively, appropriate cutoff (or threshold) values can be used to make the determination. In such methods, if the level of SgII is above an appropriate cutoff level (the “rule in” cutoff level) then this is indicative of a positive diagnosis, a severe condition or a poor prognosis. If the level of SgII is below the “rule out” cutoff level then this is indicative of the absence of a condition, a less severe condition or a good prognosis. Levels of a biomarker in between the “rule in” and “rule out” cutoff levels represent a grey area, i.e. biomarker levels where determination is uncertain and further testing is required. Appropriate methods of determining cutoff values for determining the severity of a condition or for prognosing the likely outcome of a condition are well known and documented in the art and any of these may be used (Antman E M., 2002, NEJM, 346 (26): 2079-2082, Maisel A S et al., 2002, NEJM; 347 (3): 161-168). The cutoff values may differ depending on the condition in question and the assay method used to measure SgII and thus, preferably, appropriate cutoff levels should be determined for the particular condition and the method of assay which is to be used. This can readily be done by a person skilled in the art.

In the methods of the present invention, exemplary “rule in” cutoff levels for a positive diagnosis, or determining a severe condition or a poor prognosis of a critical care condition, in particular severe sepsis or septic shock, are ≧0.20 nmol/L for SgII (or fragments thereof), and exemplary “rule out” cutoff levels are <0.15 nmol/L for SgII (or fragments thereof). SgII levels≧0.15<0.20 nmol/L thus may represent a “grey area”, i.e. biomarker levels where determination is uncertain and further testing is required. Alternative “rule in” cutoff levels for a positive diagnosis, or determining a severe condition or a poor prognosis of a critical care condition, in particular severe sepsis or septic shock, are ≧0.22 nmol/L for SgII (or fragments thereof).

However, as mentioned above, as cutoff levels may differ with the condition evaluated and the method used for measuring SgII in a sample, the levels provided herein are particularly relevant to methods in which the levels of SgII are determined by an assay (e.g. a radioimmunoassay assay) which measures the epitopes SgII154-165, e.g. as described elsewhere herein, and the determination of sepsis or septic shock. These levels are also of particular relevance to levels of these markers found in specific body fluids, e.g. as in this case, circulatory samples, e.g. blood and in particular plasma (particularly heparin plasma samples). In addition, these levels are of particular relevance in samples taken from human subjects. When SgII levels are measured in serum, the absolute levels of SgII can appear lower than the SgII levels in heparin plasma samples. An exemplary “rule in” cutoff level is 0.15 nmol/L when SgII levels are measured in serum samples. In particular, a serum level of 0.15 nmol/L is a useful “rule in” cutoff level for determining the prognosis of patients with non-septic critical illness.

Thus, in preferred embodiments of the invention, a level of SgII (or fragments thereof) measured in blood or plasma of at least 0.20 nmol/L if measured by a radioimmunoassay which measures the epitopes SgII154-165 (or an equivalent value for SgII if measured by an alternative assay, e.g. reflecting an increase in level of the biomarker of e.g. ≧10% compared to the level found in an appropriate control sample or subject) is indicative of a positive diagnosis, a severe condition or a poor prognosis.

Thus, in additional preferred embodiments of the invention, a level of SgII (or fragments thereof) measured in blood or plasma of less than 0.15 nmol/L if measured by a radioimmunoassay which measures the epitopes SgII154-165 (or an equivalent value for SgII if measured by an alternative assay, e.g. reflecting a level of the biomarker of e.g. 10% or more below what is found in an appropriate control sample or subject) is indicative of the absence of disease, a less severe condition or a good prognosis.

The level of SgII, or fragments thereof, in a subject is indicative of the severity of a critical illness, with the level of SgII (or fragments) increasing with increased severity of critical illness. Thus, an increase in level is indicative of the severity of condition and the more increased the level of SgII (or fragments), the greater the likelihood of a more severe form of critical illness and possibly death.

Serial (periodical) measuring of SgII, or fragments thereof, may also be used to monitor the severity of critical illness or to determine prognosis of critical illness, looking for either increasing or decreasing levels over time. As high levels are shown to be associated with poorer functional status, the use of serial measurement of SgII, or fragments thereof, may also be used to guide and monitor therapy, in the situation of “watchful waiting” before treatment (e.g. pharmaceutical therapy, non-invasive or invasive ventilatory support) or surgery, or during or after treatment to evaluate the effect of treatment and look for signs of therapy failure. Measuring of SgII, or fragments thereof, may also be used to identify and diagnose specific pathophysiological processes activated in patients with critical illness, and to guide targeted therapy to reduce the effect of such detrimental processes.

For prognostic use and for monitoring of individuals with critical care conditions, there is assumed to be a linear association between severity of a condition/risk and SgII (or fragment) levels, with low risk patients or patients with less severe critical care conditions having levels close to the “rule out” cutoff limits, but with any increasing level associated with increasing severity of critical care conditions or worsening of prognosis. In addition, in general, the larger the increase the greater the severity of critical care conditions or the poorer the prognosis. For example, SgII (or fragment) levels≧0.25 nmol/L are considered to be associated with especially severe critical care conditions or an especially poor prognosis, in particular when the critical care condition concerned is severe sepsis or septic shock, or a non septic disease or condition, such as trauma, acute respiratory failure, gastrointestinal disease, intoxication, neurological conditions, any condition for which the patient requires mechanical ventilation and complications from surgery. Likewise, an increase in the level of the biomarker of ≧20%, ≧25%, ≧30% or ≧35% compared to the level found in an appropriate control sample or subject (i.e. when compared to a control level) is indicative of especially severe critical care conditions or an especially poor prognosis. Additionally, any increase≧20% (e.g. ≧20%, ≧25%, ≧30% or ≧35%) from an individual's baseline biomarker value during serial biomarker testing is considered as clearly increasing severity of a critical care condition or worsening of prognosis, even when the biomarker levels are below “rule in” cutoff levels.

Thus, in preferred embodiments of the invention, a level of SgII (or fragment) of at least 0.25 nmol/L if measured by a radioimmunoassay which measures the epitopes SgII154-165 (or an equivalent value for SgII if measured by an alternative assay, e.g. reflecting an increase in level of the biomarker of e.g. ≧20% compared to the level found in an appropriate control sample or subject) is indicative of especially severe critical care conditions or an especially poor prognosis. Such levels are particularly appropriate for circulatory samples and for the determination of severity or prognosis of severe sepsis or septic shock, or a non septic disease or condition, such as trauma, acute respiratory failure, gastrointestinal disease, intoxication, neurological conditions, any condition for which the patient requires mechanical ventilation and complications from surgery.

Although the methods of the present invention may be used to determine the clinical severity of critical care conditions as evaluated by any appropriate clinical measure, a typical and preferred measure of clinical severity is a calculated value in the upper range of the established ICU risk scores, e.g. SAPS II, SOFA, APACHE and LOD score.

In the results described herein, in the largest and most recent cohort of patients studied it has been shown that circulating levels of SgII were significantly increased (p<0.001) on hospital admission and after 72 hours (p=0.003) in non-surviving subjects (i.e. subjects that die) with severe sepsis or septic shock compared to surviving control subjects (subjects that do not die) with severe sepsis or septic shock. Such a significant increase was observed on admission in patients with both in-hospital mortality (i.e. patients which died in hospital but not necessarily in an intensive care unit) and ICU mortality (i.e. patients which died in an intensive care unit), p<0.001 and p<0.01, respectively. Thus, in both univariate and multivariate logistic regression analysis, levels of SgII on the day of admission to ICU are significantly (and independently) associated with both in-hospital mortality and mortality in the ICU. The results obtained in this study also demonstrate that if two patients have the same SAPS II and SOFA score, an SgII level above 0.17 nmol/L on hospital admission indicates a 95% and 116% increase in odds, respectively, for dying during their period in the hospital.

ROC-AUC values for determining overall prognostic accuracy of SgII for in-hospital mortality and ICU mortality were also good (AUC 0.65 and 0.69), respectively) and non-inferior to the AUCs of SOFA score and SAPS II score in the same cohort of patients.

Advantageously, therefore determination of SgII levels as described herein provides additional and incremental information to the information obtained by patient history, clinical status and established markers of risk. SgII is thus a marker with potential to provide real clinical information.

The results from the largest and most recent cohort of patients thus confirm that determining circulating SgII levels in patients admitted to hospital with sepsis and critical illness provides strong prognostic information with regard to both the likelihood of a patient dying in hospital (hospital mortality) and to the likelihood of a patient developing circulatory failure/septic shock during the hospitalization. In this regard, SgII has been shown to be superior to other biomarkers known in the art (chromogranin A, NT-proBNP and hs-cTNT) and provides incremental information to that obtained by SOFA and SAPSII scores which are necessarily obtained after at least 24 hours in hospital.

In addition, results described herein demonstrate that SgII levels measured early after hospitalization can be used to assess short-term (<30 days) and long-term prognosis in a heterogenous group of “non-septic” patients with critical illness (i.e. critically ill patients which were not suffering from sepsis).

Also described herein is a study of a smaller cohort of patients in which it has been shown that circulating levels of SgII were significantly increased (p<0.03) in non-surviving subjects (i.e. subjects that die) with severe sepsis or septic shock compared to surviving control subjects (subjects that do not die) with severe sepsis or septic shock. Such a significant increase was observed in patients with both in-hospital mortality (i.e. patients which died in hospital but not necessarily in an intensive care unit) and ICU mortality (i.e. patients which died in an intensive care unit), p=0.01 and p=0.02, respectively. In this study, ROC-AUC values for determining overall prognostic accuracy of SgII for in-hospital mortality and ICU mortality were also good (AUC 0.62 and 0.66, respectively) and similar to those for the SOFA score and SAPS II score in the same cohort of patients.

For risk stratification of an individual, either previously healthy or diseased (and indeed for all the aspects of the invention described herein), SgII, or fragments thereof, may be used individually as a unique biomarker and thus evaluated alone, or the biomarker SgII (or fragment thereof) may be used in combination, as part of a broader panel of different critical illness determination systems or markers (multimarker approach).

The methods of the invention may also be used for diagnosing, i.e. determining the presence or absence of, a condition which requires critical care. Following from the above discussion the methods of the invention can be used to identify subjects requiring more intensive monitoring or subjects which might benefit from early therapeutic intervention, e.g. by surgery, pharmaceutical therapy, or non-pharmaceutical therapy.

Thus, in a yet further aspect the present invention provides a method to identify subjects requiring more intensive monitoring or subjects which might benefit from early therapeutic intervention, said method comprising determining the level of SgII, or fragments thereof, in said subject.

The methods of the invention can also be used to monitor the progress of critical illness in a subject. Such monitoring can take place before, during or after treatment of critical illness by surgery or therapy.

Subsequent to such surgery or therapy, the methods of the present invention can be used to monitor the progress of critical illness, to assess the effectiveness of therapy or to monitor the progress of therapy, i.e. can be used for active monitoring of therapy. In such cases serial (periodic) measurement of levels of SgII, or fragments thereof, for a change in said biomarker levels will allow the assessment of whether or not, or the extent to which, surgery or therapy has been effective against the critical illness, whether or not critical illness is re-occurring or worsening in the subject and also the likely clinical outcome (prognosis) of the critical illness should it re-occur or worsen.

Equally, the methods of the present invention can be used in the active monitoring of patients which have not been subjected to surgery or therapy, e.g. to monitor the progress of the critical illness in patients where indication for starting therapy is uncertain. Examples of this may be patients with arterial hypotension that may initially be treated only with fluid resuscitation, and the use of therapy to increase blood pressure initially withheld (i.e. dopamine, norepinephrine or epinephrine infusion). In this situation a high or increasing SgII level would warrant more aggressive therapy and thus guide treatment. Similarly, by reflecting disease severity SgII measurement could be of use in a situation in which there is clinical suspicion for, but no confirmation of, a bacterial infections, and where antibiotics therapy initially may be withheld. A high or increasing SgII level in this setting would warrant more aggressive therapy and thus guide treatment, including indicating appropriateness for starting antibiotics or other supportive therapies/procedures (fluid resuscitation, pulmonary physiotherapy, bronchoscopy, echocardiography, change mechanical ventilation, etc). Again serial measurements will allow an assessment of whether or not, or the extent to which, the critical illness is worsening or another critical illness is developing, thus, for example, allowing a more reasoned decision to be made as to whether therapeutic intervention is necessary or advisable.

Generally, in such embodiments, an increase in the level of SgII, or fragments thereof, is indicative of progression of critical illness. Conversely, a decrease in level is indicative of improvement or reduced progression.

Thus, in a yet further aspect the present invention provides a method of monitoring a subject with a condition which requires critical care, said method comprising determining the level of SgII, or fragments thereof, in said subject.

Thus, the observed association of increased levels of SgII with the presence, severity and prognosis of critical illness will also allow active monitoring of patients and their treatment to take place and the tracking of clinical outcomes. Thus, the methods of the invention can be used to guide management of critical care conditions and preferably optimize therapy.

As mentioned above, the identification of quality biomarkers for critical care patients such as SgII or fragments thereof, allows a multimarker approach for determining severity or prognosis. Thus, the methods of the present invention which comprise determining the levels of SgII, or fragments thereof, might not only be used in place of other systems and biomarkers for the determination of critical illness (i.e. be used as single markers), but might also be used in combination, or in addition to the measurement of one or more other systems or biomarkers known for the determination of critical illness (i.e. in a multimarker assay). Examples of other parameters that could be combined with SgII measurement are ICU scoring models (SAPS II, SOFA, APACHE, LOD score), individual parameters of the scoring models or additional factors associated with a poor prognosis such as age, echocardiographic parameters, physiological parameters such as (invasive) blood pressure and pulmonary function (i.e. oxygen saturation, peak expiratory flow, etc), whether the patient is suffering from renal failure or other organ failure, comorbidities, standard biochemical analysis, and other cardiac biomarkers such as the B-type natriuretic peptides (BNP, NT-proBNP). However, SgII may be combined with any parameter that supplements SgII and improves the diagnostic and prognostic utility of SgII measurement alone.

A further embodiment of the invention provides the use of the methods of the invention either alone or in conjunction with other known methods for determining severity or determining prognosis of a condition which requires critical care.

A yet further aspect provides a kit for diagnosing or for determining severity or prognosis of a critical care condition, which comprises an agent suitable for determining the level of SgII, or fragments thereof, in a sample. Preferred agents are antibodies directed to SgII, or fragments thereof. Other preferred agents are labelled SgII molecules, or fragments thereof. In preferred aspects said kits are for use in the methods of the invention as described herein.

Thus, in the present invention it can be seen that it has been recognised that increased, elevated, or generally high levels of SgII, or fragments thereof, are markers of the presence of, the severity of, or future outcome (prognosis) of conditions which require critical care and in particular severe sepsis or septic shock. Thus, measurement of SgII in critically ill patients, in particular in ICU patients, can enhance the early identification of patients of high risk thus improving both therapy and monitoring of these subjects. Reference herein to “high” levels of SgII, or fragments thereof, includes a level of ≧0.15 nmol/L, ≧0.20 nmol/L or ≧0.25 nmol/L, particularly in a circulatory sample such as blood, serum or plasma.

Reference herein to “SgII” includes reference to all forms of SgII which might be present in a subject (naturally occurring SgII molecules), including derivatives, mutants and analogs thereof (e.g. naturally occurring variants), in particular fragments thereof or modified forms of SgII or their fragments. Exemplary and preferred modified forms include forms of these molecules which have been subjected to post translational modifications such as glycosylation or phosphorylation.

As discussed above, SgII are pro-peptides with multiple recognition sites for endopeptidases. Thus, in the methods of the invention described herein, any fragments of SgII, in particular naturally occurring fragments, especially those found in body fluids, can be analyzed as an alternative to SgII itself (full length SgII). Examples of such fragments are described in the art, and a particularly preferred fragment is secretoneurin (SN), which is a small 33 amino acid peptide (Taupenot L et al. New Engl J Med 2003; 348:1134-1149), although it is quite possible that other fragments will be identified in the future.

For SgII, preferred fragments are those containing the SgII epitope corresponding to amino acid residues 154-165 of SgII or 172-186 of SgII, for example SN.

The amino acid sequence of SgII without its signal sequence is outlined below and the amino acid residues of the fragments of SgII as described herein can be determined with reference to this sequence.

SgII
SFQRNQLLQKEPDLRLENVQKFPSPEMIRALEYIENLRQQAHKEESSPD
YNPYQGVSVPLQQKENGDESHLPERDSLSEEDWMRIILEALRQAENEPQ
SAPKENKPYALNSEKNFPMDMSDDYETQQWPERKLKHMQFPPMYEENSR
DNPFKRTNEIVEEQYTPQSLATLESVFQELGKLTGPNNQKRERMDEEQK
LYTDDEDDIYKANNIAYEDVVGGEDWNPVEEKIESQTQEEVRDSKENIE
KNEQINDEMKRSGQLGIQEEDLRKESKDQLSDDVSKVIAYLKRLVNAAG
SGRLQNGQNGERATRLFEKPLDSQSIYQLIEISRNLQIPPEDLIEMLKT
GEKPNGSVEPERELDLPVDLDDISEADLDHPDLFQNRMLSKSGYPKTPG
RAGTEALPDGLSVEDILNLLGMESAANQKTSYFPNPYNQEKVLPRLPYG
AGRSRSNQLPKAAWIPHVENRQMAYENLNDKDQELGEYLARMLVKYPEI
INSNQVKRVPGQGSSEDDLQEEEQIEQAIKEHLNQGSSQETDKLAPVSK
RFPVGPPKNDDTPNRQYWDEDLLMKVLEYLNQEKAEKGREHIAKRAME
NM

The “increase” in the levels or “increased” level of SgII, or fragments thereof, as described herein includes any measurable increase or elevation of the marker in question when the marker in question is compared with a control level. Said control level may correspond to the level of the equivalent marker in appropriate control subjects or samples, e.g. may correspond to a cutoff level (or threshold level) or range found in a control or reference population. Alternatively, said control level may correspond to the level of the marker in question in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements of SgII in individuals, are taken looking for changes in the levels of SgII. In this regard, an appropriate control level will be the individual's own baseline, stable, nil, previous or dry value (as appropriate) as opposed to a control or cutoff level found in the control population. Control levels may also be referred to as “normal” levels or “reference” levels. The control level may be a discrete figure or a range. In addition, as mentioned above, such comparison with a control level, would not generally involve carrying out active tests on control subjects as part of the methods of the present invention but would generally involve a comparison with a control level which had been determined previously from control subjects and was known to the person carrying out the methods of the invention.

As will be clear from the discussions herein, the methods of the present invention can involve single or one off measurements or determinations of the level of SgII in a subject, or may involve multiple measurements or determinations over a period of time, e.g. for the ongoing monitoring of critical care conditions.

Preferably the increase in level will be significant, more preferably clinically or statistically significant, most preferably clinically and statistically significant.

Methods of determining the statistical significance of differences in levels of a particular biomarker are well known and documented in the art. For example herein an increase in level of a particular biomarker is generally regarded as significant if a statistical comparison using a significance test such as a Student t-test, Mann-Whitney U Rank-Sum test, chi-square test or Fisher's exact test, as appropriate, shows a probability value of <0.05 or <0.10. More detail on appropriate methods of statistical analysis is provided in the Examples.

However, ideally any test also needs to be of clinical value. To test the discriminatory ability of a biomarker to distinguish between two different sets of subjects the test of choice is considered to be the area under the receiver-operating characteristic curve (ROC-AUC). With ROC-AUC you get a measurement of sensitivity and specificity for a biomarker across the entire spectrum of cutoff values, and tests with good ROC-AUCs are considered possibly clinically important. ROC-AUC can be used both for evaluating diagnostic and prognostic merit of a test. In addition, both logistic regression analysis or Cox proportional hazards regression analysis may be used for evaluating prognostic merit.

Put in simple terms, for a prognostic assay, a ROC-AUC value of, for example, 0.65 or 0.62 for SgII, with in-hospital mortality as an end point, indicates that when comparing two patients with a critical care condition, in particular severe sepsis or septic shock, there is a probability of 65% or 62%, respectively that the SgII level will be higher in the patient subsequently dying during hospitalization compared to the patient surviving the hospitalization. For SgII, with ICU mortality as an end point, the ROC-AUC values have been shown to be, for example, 0.69 or 0.66, meaning that the corresponding probability is 69% or 66%, respectively, for a higher SgII level in a patient with a critical care illness that subsequently dies in the ICU compared to an similar patient with a critical illness that survives the ICU. SgII as a marker has non-inferior sensitivity and specificity for risk estimation when compared to the current gold-standard tests of SAPS II and SOFA.

The “decrease” in the levels or “decreasing” level, or “lower” level or “lowering” of the level of SgII, or fragments thereof, as described herein includes any measurable decrease or reduction of the marker in question when the marker in question is compared with a control level. Said control level may correspond to the level of the equivalent marker in appropriate control subjects or samples. Alternatively and preferably, said control level may correspond to the level of the marker in question in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements of SgII in individuals are taken looking for changes in the levels of SgII. In this regard, an appropriate control level will be the individual's own baseline, stable, nil, previous or dry value (as appropriate) as opposed to a control level found in the control population. The control level may be a discrete figure or a range. In addition, as mentioned above, such comparison with a control level, would not generally involve carrying out active tests on control subjects as part of the methods of the present invention but would generally involve a comparison with a control level which had been determined previously from control subjects and was known to the person carrying out the methods of the invention.

Preferably the decrease in level will be significant, more preferably clinically or statistically significant, most preferably clinically and statistically significant.

Methods of determining the statistical significance of differences in levels of a particular biomarker are well known and documented in the art. For example herein a decrease in level of a particular biomarker is generally regarded as significant if a statistical comparison using a significance test such as a Student t-test, Mann-Whitney U Rank-Sum test, chi-square test or Fisher's exact test, as appropriate, shows a probability value of <0.05 or <0.10. More detail on appropriate methods of statistical analysis is provided in the Examples.

Appropriate control subjects, samples or control levels for use in the methods of the invention would be readily identified by a person skilled in the art. Such subjects or levels might also be referred to as “normal” subjects or levels or as a reference population or level. Examples of appropriate control subjects would include healthy subjects, for example, individuals who have no history of any form of disease and do not use any medication on a regular basis, and found to have a normal clinical evaluation by a physician. Additional control groups may be patients suffering from similar conditions as the patient being evaluated, but not in critical illness, i.e. not with life threatening disease. Examples of this may be patients with pneumonia and trauma patients, but with mild disease and not considered to have a life-threatening critical illness. Alternative control samples or levels may be obtained from subjects who have suffered from critical illness and survived or recovered. The levels of SgII, or fragments thereof, found in such patients can be used as a threshold or control level for comparison to the levels found in a subject with a critical illness. Levels of SgII in critically ill patients which are significantly increased over such control levels can be indicative of severe disease or a poor prognosis. Thus, such controls are particularly useful where methods of prognosis or determining severity of illness are concerned.

The level of SgII or fragments thereof, in a subject can be determined by analysis of any appropriate sample from the subject, for example a tissue sample, a circulatory sample such as blood (e.g. serum or plasma) or from other easily accessible body fluids (e.g. urine, saliva). Levels are generally lower in saliva and urine than the corresponding circulating levels but these levels can still be determined. Preferred samples are body fluids.

Reference herein to “body fluid” includes reference to all fluids derived from the body of a subject. Exemplary fluids include blood (including all blood derived components, for example plasma, serum, etc) urine, saliva, tears, bronchial secretions or mucus. Preferably, the body fluid is a circulatory fluid (especially blood or a blood component), urine or saliva. An especially preferred body fluid is blood or a blood component, in particular plasma or serum, especially plasma. Another especially preferred body fluid is saliva. A particularly preferred sample to be analysed is blood (e.g. serum or plasma).

Levels of SgII, or fragments thereof, in a sample, e.g. in a sample of body fluid, e.g. in a blood, serum, plasma, urine or saliva sample, or in tissue samples, can be measured by any appropriate assay, a number of which are well known and documented in the art and some of which are commercially available. The level of SgII, or fragments thereof, in a sample, e.g. a circulatory sample, other body fluid sample or tissue sample can be measured at the gene level by measuring the levels of nucleic acids (e.g. DNA or RNA) encoding SgII (or a fragment thereof), for example by RT-PCR or qRT-PCR, or at the protein level, e.g. by immunoassay such as a radioimmunoassay (RIA) or fluorescence immunoassay, immunoprecipitation and immunoblotting or Enzyme-Linked ImmunoSorbent Assay (ELISA), with RIA and/or ELISA normally being the method of choice.

Preferred assays are those which can be carried out at the point of treatment or at the bedside of the patient. Preferred assays are ELISA-based assays, although RIA-based assays, such as those described in Stridsberg et al., 2008 (Reg Peptides; 148: 95-98), can also be used very effectively. Both ELISA- and RIA-based methods can be carried out by methods which are standard in the art and would be well known to a skilled person. Such methods generally involve the use of an antibody to SgII, or fragments thereof, which is incubated with the sample to allow detection of SgII (or fragments thereof) in the sample. Any appropriate antibodies can be used and examples of these are described elsewhere herein and in the prior art. For example, appropriate antibodies to SgII, or antibodies which recognise particular epitopes of SgII, can be prepared by standard techniques, e.g. by immunization of experimental animals as described in Stridsberg et al., 2008, supra). The same antibodies to SgII, or fragments thereof, can generally be used to detect SgII (or fragments) in either a RIA-based assay or an ELISA-based assay, with the appropriate modifications made to the antibodies in terms of labelling etc., e.g. in an ELISA assay the antibodies would generally be linked to an enzyme to enable detection. Any appropriate form of assay can be used, for example the assay may be a sandwich type assay or a competitive assay.

In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluoresce so that the amount of antigen in the sample can be determined through the magnitude of the fluorescence. For RIA, a known quantity of an antigen is made radioactive, frequently by labeling it with gamma-radioactive isotopes of iodine attached to tyrosine. This radiolabeled antigen is then mixed with a known amount of antibody for that antigen, and as a result, the two chemically bind to one another. Then, a sample from a patient containing an unknown quantity of that same antigen is added. This causes the unlabeled (or “cold”) antigen from the sample to compete with the radiolabeled antigen for antibody binding sites. As the concentration of “cold” antigen is increased, more of it binds to the antibody, displacing the radiolabeled variant, and reducing the ratio of antibody-bound radiolabeled antigen to free radiolabeled antigen. The bound antigens are then separated from the unbound ones, and the radioactivity of the free antigen remaining in the supernatant is measured. A binding curve can then be plotted, and the exact amount of antigen in the patient's serum can be determined. Measurements are usually also carried out on standard samples with known concentrations of marker (antigen) for comparison.

A preferred assay for SgII currently being employed is a radioimmunoassay using antibodies to measure the epitope SgII 154-165 (e.g. as described in Stridsberg M et al., 2008, supra). This RIA method measures all fragments (short or long) that have the aforementioned epitopes. Such assays thus measure SgII and any fragments which include the relevant epitopes. The results presented herein thus also identify the fragment SgII 154-165 to be important fragments/epitopes in critical illness. However, this in no way excludes a role for other SgII fragments in critical illness conditions.

For assays involving the use of SgII antibodies, appropriate antibodies are commercially available for immunoblotting and immunohistochemistry (e.g. secretoneurin antibody from Phoenix Pharmaceuticals, Burlingame, Calif., U.S.A). The use of immunoblotting is however less preferred for measuring levels of SgII as it is much less practical in patient management due to it being semi-quantitative, too time consuming (approximately 36 hours) and requiring expertise technical knowledge of the method. Immunohistochemistry is a method only for use on solid tissue, and thus this method is not appropriate for embodiments where levels in body fluids are measured.

If plasma (or some other blood component) is the sample to be analysed, then prior to the assay, plasma (or the other blood component) can be separated from a blood sample by methods well known and documented in the art.

As also described above, if tissue samples rather than body fluid samples are to be analysed, then again the levels of SgII, or fragments thereof, can readily be analysed at the gene level or protein level for example by preparing appropriate samples from appropriate tissues, by methods well known and described in the art. In addition, for example, immunohistochemistry with appropriate antibodies as set out above could be carried out on tissue sections.

Although the diagnostic methods of the invention are generally carried out in vitro, in other embodiments of the invention in vivo methods might be used. Thus, yet further aspects are methods of imaging of a subject comprising the administration of an appropriate amount of a binding entity (e.g. an antibody or other binding protein) which can target SgII, or fragments thereof, to the subject and detecting the presence and/or amount and/or the location of the binding entity in the subject. Such methods can thus be used in the imaging of subjects which have a condition which requires critical care. Such methods can also be used to monitor the progress of critical care conditions or for monitoring therapy of critical care conditions.

For such methods of imaging, any appropriate binding entity can be used, e.g. any entity which has the ability to bind to SgII, or fragments thereof, in vivo. Preferred binding entities are antibodies or antibody fragments. Antibodies to SgII, or fragments thereof, are described in the art and some are described specifically herein. Any of these can be used. Alternatively, as discussed above, appropriate antibodies can readily be generated by the skilled man using methods well known and documented in the art. Preferred antibodies or binding entities are those that bind to the epitopes SgII 154-165, SgII 172-186, which are believed to be important in critical illness. In such methods, the binding entity, preferably the antibody, may be labeled with any marker which is detectable in vivo (an in vivo detectable label or imaging agent/modality), preferably using non-invasive methods.

Many appropriate in vivo detectable labels or imaging agents/modalities are known in the art, as are methods for their attachment to binding entities and antibodies. Such detectable labels allow the presence, amount or location of binding entity-target (in this case binding entity-SgII) complexes in the subject to be examined.

Specific examples of imaging agents/modalities which might be used are a radio-opaque or radioisotope such as ³hydrogen, ¹⁴-carbon, ³²phosphorus, ³⁵sulphur, ¹²³iodine, ¹²⁵iodine, ¹³¹iodine, ⁵¹chromium, ³⁶chlorine, ⁵⁷cobalt, ⁶⁷cobalt, ⁶⁷copper, ¹⁵²Eu, ⁶⁷gallium, ¹¹¹indium, ⁵⁹iron, ¹⁸⁶rhenium, ¹⁸⁸rhenium, ⁷⁵selenium, ^{99m}technetium and ⁹⁰yttrium; metal ions (for example paramagnetic ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III) or other metal ions such as lanthanum (III), gold (III), lead (II), and bismuth (III)); fluorescent (fluorophore) compounds, such as fluorescein, rhodamine or luciferin.

Thus, additionally, the invention also provides the use of SgII, or fragments thereof, as molecular targets in tissue for imaging modalities when investigating critical illness in an individual. The use of upregulated proteins as specific targets in disease processes is expected to increase during the next decade, and improve the clinical usefulness of all imaging modalities, e.g. MRI, CT scanning, SPECT, tissue echocardiography, among a few. As SgII levels clearly are increased in the circulation in patients with a critical illness, SgII production is increased in subjects with critical illness. Using SgII as a molecular target in imaging in patients with critical illness could thus provide identification of specific organs and tissues that have increased SgII production, also diagnosing dysfunction in these organs.

This method may also be used for identifying or diagnosis of activated pathophysiological axes and pathways, and for monitoring of therapy in individuals with critical illness (e.g. both subclinical and overt disease) as described in more detail elsewhere herein. Again as described in more detail elsewhere herein, more specifically, the epitopes SgII 154-165 and SgII 172-186 have been identified as important molecular targets in a subject for imaging modalities in evaluating critical illness in general, and sepsis, severe sepsis and septic shock especially.

The methods of the invention as described herein can be carried out on any type of subject which is capable of suffering from conditions which require critical care. The methods are generally carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs). Subjects may also be referred to herein as patients. If a non-human subject is involved then non-human forms of SgII will need to be determined as appropriate depending on the species of the subject.

In preferred embodiments the mammals are humans. However, in other embodiments, SgII, or fragments thereof, can be used as markers of critical illness in any appropriate animal model. Thus, in such embodiments, the methods of the invention can be carried out on any appropriate experimental animal model used for investigating critical illness or any aspect of critical care. Such methods can be used to test and identify (screen) potential new therapeutic agents (drugs and non-drugs) for critical illness, e.g. a test substance can be administered to the animal model and the effect on SgII levels, or fragments thereof, analysed.

Thus, a preferred embodiment of the present invention provides a method of testing the therapeutic potential of a substance for the treatment of critical illness, comprising administering a test substance to an experimental animal suffering from a critical illness and determining the level of SgII, or fragments thereof, in said animal.

In such methods, for example, a decrease or lowering of SgII levels would be indicative of a possible therapeutic effect or therapeutic potential.

Once a substance with appropriate therapeutic potential has been identified, said substance is a candidate for use in therapy. Thus, the present invention also provides methods in which the substances identified are manufactured and optionally formulated with at least one pharmaceutically acceptable carrier or excipient. The present invention also provides methods in which the substances identified, manufactured or formulated are used in the treatment of critical illness.

The methods of the invention can also be used in conjunction with animal models to test or investigate the molecular mechanisms behind critical illness and to investigate aspects of signalling pathways involved in critical illness.

Appropriate animal models for use in these aspects would be well known to a person skilled in the art and would include any animal model which can be used to study critical illness, in particular severe sepsis or septic shock. Relevant models in all species would include sepsis models in mouse, rat, pig, and dog, with sepsis either induced by injecting microbiological material itself or by injecting microbiological material known to illicit a sepsis like response, i.e. lipopolysaccharides (LPS) or any other known endotoxin or exotoxin. Similarly, trauma animal models, models of burn injury, and other experimental animal models for conditions generally considered to induce critical illness may be used. Appropriate models are also described in “A Practical Approach to Animal Models of Sepsis”, Sourcebook of Models for Biomedical Research, 2008, pages 473-482.

Preferred types of critical illness for these methods of testing and investigation are as described elsewhere herein.

Instead of testing on animal models in vivo, such tests could also be carried out in vitro using appropriate cell or tissue models. Examples of this may be testing responses ex vivo in cell lines to burn injury and microbiological substances associated with sepsis.

The invention will be further described with reference to the following non-limiting Examples with reference to the following drawings in which:

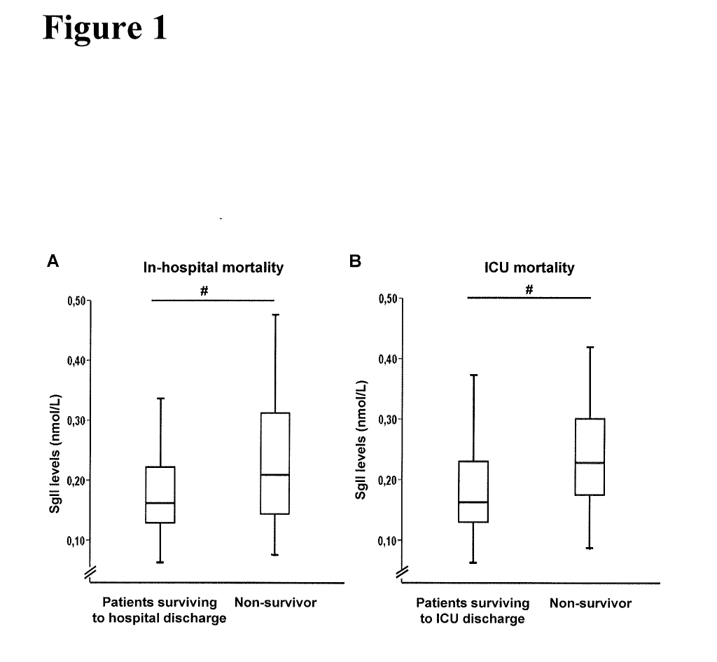

FIG. 1 shows SgII levels stratified according to in-hospital and ICU mortality.

FIG. 1A shows that circulating levels of SgII on ICU admission were significantly higher in hospital non-survivors than hospital survivors: 0.23±0.02 vs. 0.18±0.01 nmol/L, p=0.01. FIG. 1B shows SgII levels on admission to ICU were higher in patients not surviving to ICU discharge (non-survivors) compared to the patients surviving to discharge from the ICU: 0.23±0.02 vs. 0.19±0.01 nmol/L, p=0.02. The horizontal line within the box represents the median level, the boundaries of the box the 25th and 75th percentile levels, and the whiskers range (maximum value restricted to 1.5×interquartile range from the median). #p<0.05

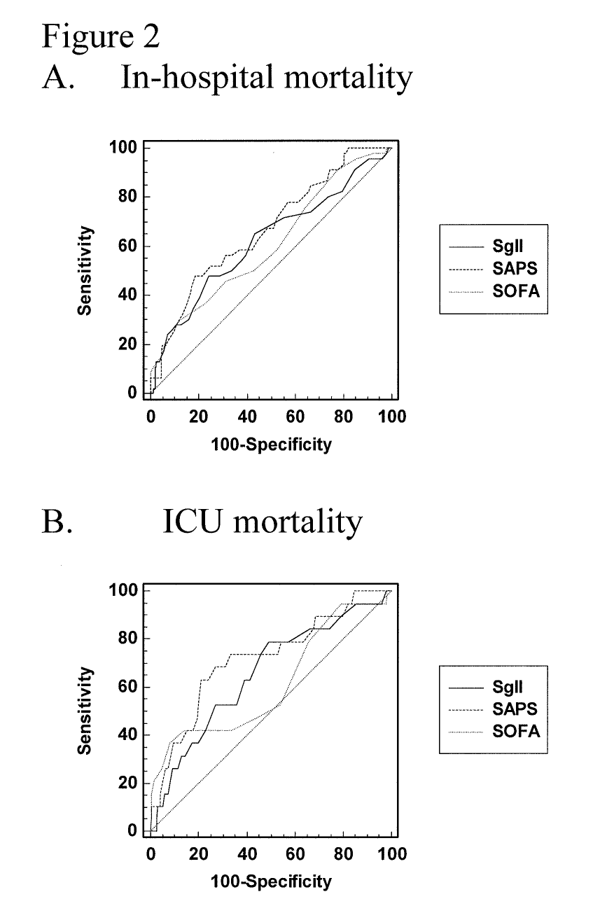

FIG. 2 shows diagnostic accuracy evaluated by receiver operating characteristics (ROC) curve analyses.

FIG. 2A shows assessment of the overall prognostic accuracy for in-hospital mortality and reveals similar AUCs for SgII levels (AUC 0.62 (95% confidence intervals 0.55-0.69)), SOFA score (AUC 0.61 (95% CI 0.54-0.68, p=0.84 vs. SgII), and SAPS II score (AUC 0.67 (95% CI 0.60-0.73, p=0.47 vs. SgII). FIG. 2B shows that SgII levels (AUC 0.66 (95% CI 0.59-0.72) were of similar strength as SAPS II (AUC 0.72 (95% CI 0.65-0.79, p=0.50 vs. SgII) and SOFA score (AUC 0.62 (95% CI 0.54-0.68, p=0.68 vs. SgII) for predicting ICU mortality.

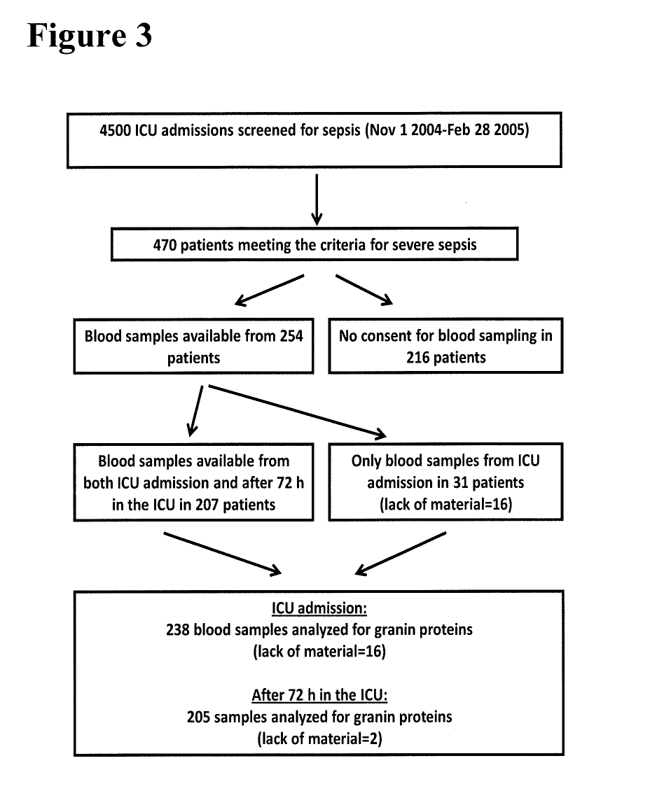

FIG. 3 is a flow chart of the study described in Example 2

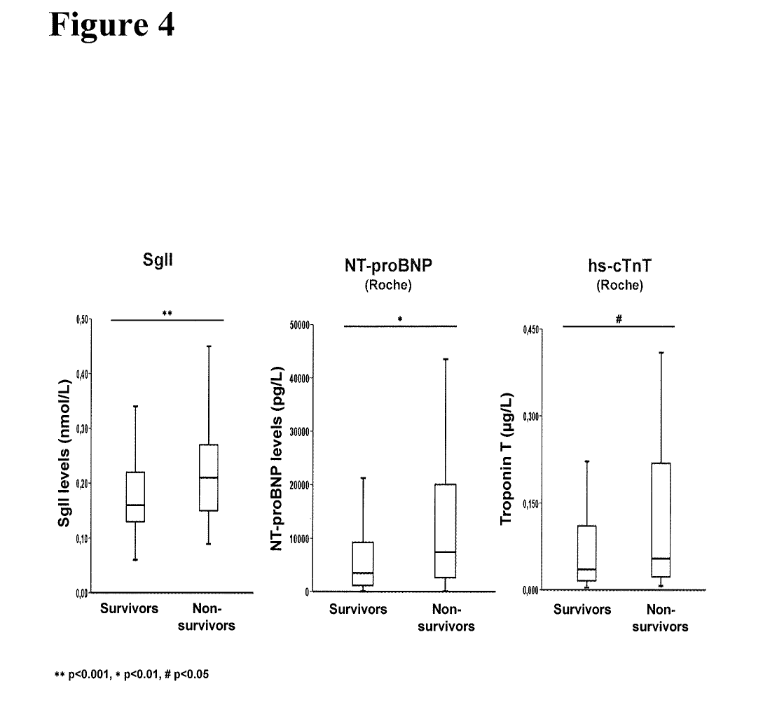

FIG. 4 shows SgII, NT-proBNP and hs-cTnT levels on inclusion in the study in patients surviving to hospital discharge and in patients not surviving to hospital discharge (non-survivors). The horizontal line within the box represents the median level, the boundaries of the box quartiles 1-3, and the whiskers range (maximum value restricted to 1.5×interquartile range from the median).

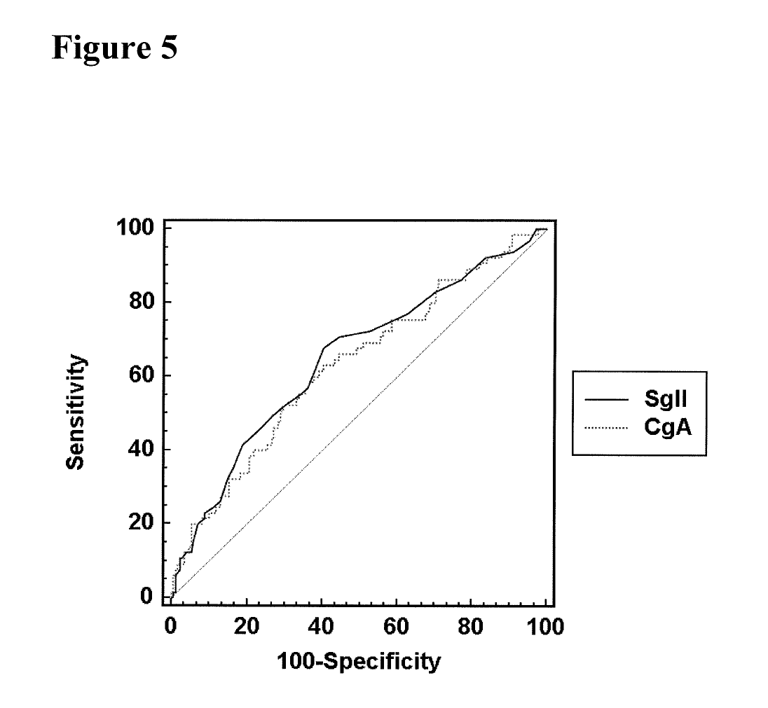

FIG. 5 shows prognostic accuracy evaluated by receiver operating characteristics (ROC) curve analysis. This data shows that SgII levels measured during the first hours after hospitalization for severe sepsis were superior to CgA levels for prognosis (AUC: 0.65 vs. 0.62).

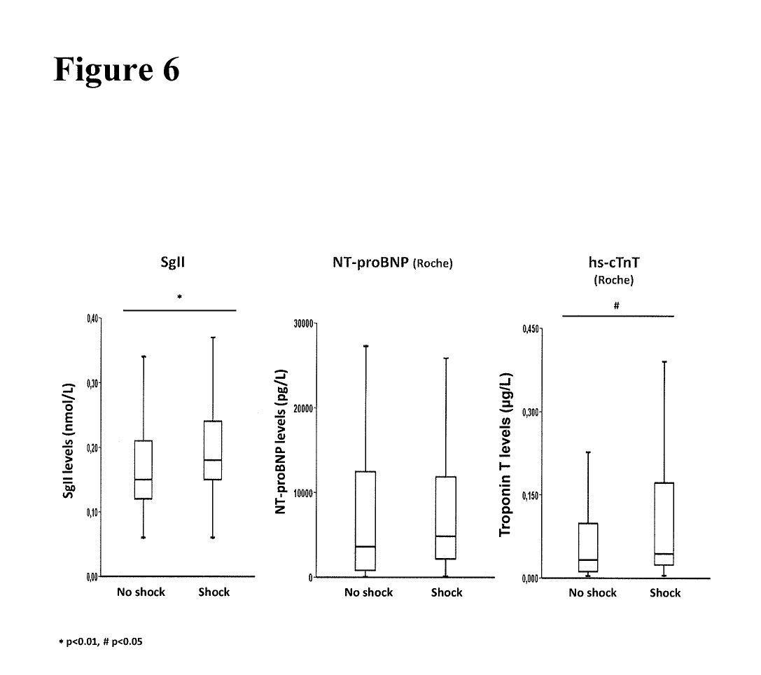

FIG. 6 shows SgII, NT-proBNP and hs-cTnT levels on inclusion in the study in patients developing circulatory failure (medical shock) during the hospitalization vs. patients not developing shock. The horizontal line within the box represents the median level, the boundaries of the box quartiles 1-3, and the whiskers range (maximum value restricted to 1.5×interquartile range from the median).

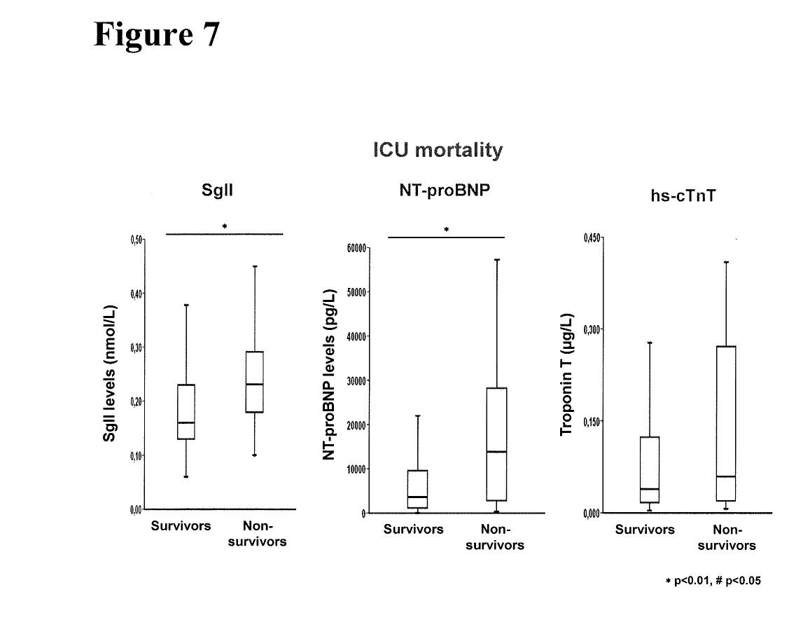

FIG. 7 shows SgII, NT-proBNP and hs-cTnT levels measured on inclusion in the study in patients surviving to ICU discharge and in patients not surviving to ICU discharge (non-survivors). The horizontal line within the box represents the median level, the boundaries of the box quartiles 1-3, and the whiskers range (maximum value restricted to 1.5× interquartile range from the median).

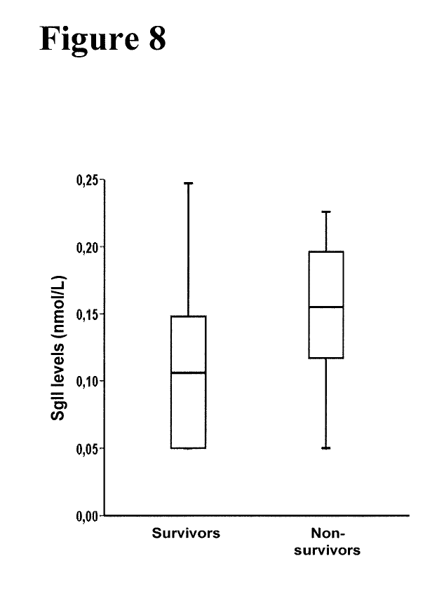

FIG. 8 shows SgII levels after 48 hrs in mechanically ventilated non-septic patients with critical illness divided into patients surviving >30 days (survivors) and patients surviving ≦30 days (non-survivors). The horizontal line within the box represents the median level, the boundaries of the box quartiles 1-3, and the whiskers range (maximum value restricted to 1.5× interquartile range from the median).

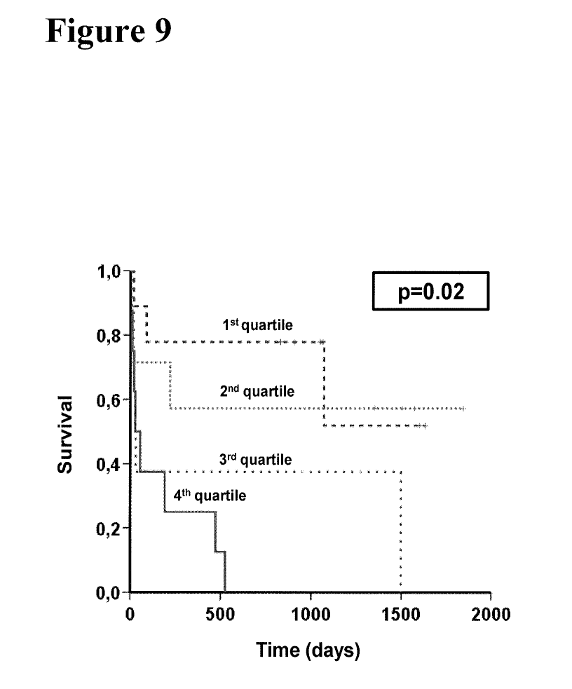

FIG. 9 shows the long-term prognosis in mechanically ventilated non-septic patients requiring mechanical ventilation divided by SgII quartiles on hospital admission.

### EXAMPLE 1

### SgII Provides Independent Information for Prognosis in Patients with Severe Sepsis and Septic Shock: Results from the FINNSEPSIS Study

This study aims to assess whether measurement of SgII levels in patients admitted to intensive care units is a useful prognostic marker in patients with severe sepsis or septic shock.

### Methods

### Study Design

The current investigation is a substudy of FINNSEPSIS, a prospective observational cohort study of the incidence and prognosis of sepsis in 24 intensive care units (ICUs) in Finland (Karlsson S, et al., Intensive Care Med. 2007; 33:435-443). In brief, all patients aged 18 years or older admitted to the ICUs of participating centres were screened daily for the American College of Chest Physicians/Society of Critical Care Medicine criteria of severe sepsis or septic shock. All patients fulfilling the criteria, i.e. patients with known or suspected infection, with two or more criteria for systemic inflammatory response syndrome, and at least one new sepsis-induced organ failure were included. In-hospital mortality, e.g. patients dying while hospitalized, and Intensive Care Unit (ICU) mortality, e.g. patients dying while still being treated in the ICU, were classified as the co-primary endpoint.

Basic hemodynamic and routine biochemical laboratory test data, as well as information concerning the use of vasoactive drugs and amount of fluids administered, were collected on a daily basis in the ICU. After the first 24 hours in the ICU, the Simplified Acute Physiology Score (SAPS) II was filled out (Le Gall Jr, et al., JAMA. 1993; 270:2957-2963). Sequential Organ Failure Assessment (SOFA) score was subsequently scored daily (Vincent J L, et al., Intensive Care Med. 1996; 22:707-710). Patient records were used to check for cardiovascular comorbidity, defined as a prior diagnosis of heart failure or coronary artery disease and the requirement for medication or a history of a disease-related procedure. All data were collected and stored via the internet to the Finnish intensive care quality consortium (Intensium, Kuopio, Finland). In-hospital and ICU mortality were recorded.

### Biochemical Analyses

Blood samples were obtained from indwelling arterial catheters or by venipuncture on admission to the ICU and again 72 hrs later. The samples were centrifuged at room temperature and the serum component was aspirated and frozen at −20° C. at individual centers. Within 3 months of collection, plasma samples were shipped on dry ice to the Helsinki University Hospital for storage at −80° C. for 4 years pending analysis. SgII analyses were performed if consent for blood sampling was obtained and serum was available.

SgII levels were measured as previously reported by an in-house made radioimmunoassay (RIA) with the epitope mapping to the secretoneurin (SN fragment) of SgII (SgII154-165) (Stridsberg M, et al., Regul Pept. 2008; 148:95-98), chromogranin B (CgB) with a RIA mapping to CgB 439-451 (Stridsberg M, et al., Regul Pept. 2005; 125:193-199), while hs-cTnT levels were determined on a cobas e 411 (Roche Diagnostics, Germany) with a commercially available assay (Elecsys Troponin T—high sensitive assay). The laboratory personnel were blinded to clinical information of the patients.

### Statistical Analyses

Data are presented as mean±SEM or as absolute values and percentages. Between group differences were assessed by the Mann-Whitney U test and Fisher exact test for continuous and categorical data, respectively. Association between variables and trends were assessed by Spearman rank correlation. Logistic regression analysis was used to assess the prognostic values of variables. Because of skewed distributions, SgII and hs-cTnT values were entered as quartiles. Other factors included in univariate analysis were age, sex, a prior diagnosis of diabetes mellitus or hypertension; SAPS II and SOFA scores; platelet count, serum creatinine and peak lactate value (both logarithmically transformed); and dose of dopamine, dobutamine, and norepinephrine infusion. Factors significantly associated with in-hospital or ICU mortality in univariate analysis were entered in multivariate analysis. A forward selection procedure was used for multivariate models. Model fit was assessed by the Hosmer-Lemeshow goodness-of-fit test. Receiver operating characteristics (ROC) curve analyses was used for evaluating prognostic accuracy. AUCs (area under the curves) were compared by the method of Hanley and McNeil (Hanley J A, et al.; Radiology. 1983; 148:839-43). P-values<0.05 were considered significant for all analyses. Statistical analyses were performed with SPSS for Windows version 14.0 (SPSS, Chicago, Ill.) with the exception of the ROC curve analysis which was performed with MedCalc for Windows, version 9.5.1.0 (MedCalc Software, Mariakerke, Belgium).

### Results

### Patient Characteristics

Samples for SgII analyses were available from 205 out of 470 patients from the FINNSEPSIS study cohort. Main reasons for non-participation were inability to provide informed consent and lack of serum. Concerning clinical characteristics and the case fatality rate, no major differences were evident between the current cohort and the complete cohort (data not shown) (Varpula M, et al., Crit Care Med. 2007; 35:1277-1283). In total 46 (22%) patients died during the hospital stay, of whom 19 (9%) died in the ICU. Characteristics of participating patients, stratified according to in-hospital survival status, are summarized in Table 1.

### SgII Levels on Admission and After 72 Hours in the ICU

Circulating SgII levels were detectable in all patients both on admission to the ICU and after 72 hours in the ICU. On admission, SgII levels ranged from 0.06 nmol/L to 0.56 nmol/L (mean±SEM: 0.19±0.01 nmol/L). Corresponding SgII values after 72 hrs concentrations were 0.05-0.52 nmol/L (0.17±0.01 nmol/L). SgII levels on admission and after 72 hours in the ICU were closely correlated (r=0.62, p<0.001), as were admission SgII and CgB levels (r=0.57, p<0.001). In contrast, there was no significant correlation found between admission SgII levels and age or hsTnT, creatinine, and lactate levels. Furthermore, SgII levels were not significantly correlated with the ICU severity models SOFA or SAPS II score calculated on day one in the ICU, or the dose of dopamine, dobutamine, and norepinephrine infusion during the ICU stay.

### Association Between SgII Levels and In-Hospital Mortality

Circulating levels of SgII on ICU admission were significantly higher in hospital non-survivors than hospital survivors: 0.23±0.02 vs. 0.18±0.01 nmol/L, p=0.01 (FIG. 1A); while levels measured after 72 hours were of borderline significance: 0.19±0.01 vs. 0.16±0.01 nmol/L, p=0.07. In contrast, admission CgB levels were not significantly different between patients dying in hospital and patients surviving to discharge (1.55±0.08 vs. 1.44±0.05 nmol/L (p=0.20). Furthermore, hs-cTnT levels were only of borderline differences for patients dying during hospitalization compared to the other patients (536±308 vs. 131±22 μg/L, p=0.06).

By univariate logistic regression analysis (Table 2), SgII levels on admission were associated with in-hospital mortality (odds ratio (OR) per one quartile increase in SgII levels: 1.50 (95% confidence interval (CI) 1.10-2.05), p=0.01). In multivariate analysis (Table 2), adjusting for the other variables significantly associated with in-hospital mortality in univariate analysis, SgII levels on admission were still clearly associated with in-hospital mortality: OR 1.54 (95% CI 1.11-2.16), p=0.01. Increasing age (OR per 1 y increase: 1.03 (95% CI 1.01-1.06), p=0.02), and SAPS II score (OR per 1 point increase: 1.04 (95% CI 1.01-1.07), p=0.01) were also independently associated with in-hospital mortality. Model fit for the final model was acceptable as evaluated by the Hosmer-Lemeshow goodness-of-fit test (p=0.25).

Assessment of the overall prognostic accuracy for in-hospital mortality, as estimated by receiver-operating characteristics analysis, revealed similar AUCs for SgII levels (AUC 0.62 (95% confidence intervals 0.55-0.69)), SOFA score (AUC 0.61 (95% CI 0.54-0.68, p=0.84 vs. SgII), and SAPS II score (AUC 0.67 (95% CI 0.60-0.73, p=0.47 vs. SgII) (FIG. 2A).

### Association Between SgII Levels and ICU Mortality

Similar to what was found for in-hospital mortality, SgII levels on admission to ICU were higher in patients not surviving to ICU discharge (non-survivors) compared to the patients surviving to discharge from the ICU: 0.23±0.02 vs. 0.19±0.01 nmol/L, p=0.02 (FIG. 1B). Admission SgII levels were significantly associated with ICU mortality in univariate analysis (OR per one quartile increase: 1.70 (95% CI 1.07-2.72), p=0.03), Table 3. SgII levels (AUC 0.66 (95% CI 0.59-0.72) were of similar strength as SAPS II (AUC 0.72 (95% CI 0.65-0.79, p=0.50 vs. SgII) and SOFA score (AUC 0.62 (95% CI 0.54-0.68, p=0.68 vs. SgII) for predicting ICU mortality (FIG. 2B).

### CONCLUSION

The novel findings of the current study of a large representative and contemporary cohort of patients with severe sepsis or septic shock are that (1) detectable levels of circulating SgII are present in all patients both on admission to the ICU and after 72 hours in the ICU; (2) SgII is not closely correlated with established factors of risk in severe sepsis and septic shock reflecting that SgII measures processes currently not recognized by the conventional risk parameters; (3) SgII levels are increased in the patients with poor prognosis and provides additional information for both ICU and in-hospital mortality to the information obtained from patient history, clinical parameters, ICU severity scoring models, and conventional cardiac biomarkers, and (4) SgII levels were also clearly superior to CgB levels, another member of the granin family, and to hs-cTnT, an established cardic biomarker, for identification of patients with critical illness, and thus also the patients with the worst prognosis. SgII thus represents a new and important biomarker in patients admitted to the ICU, and especially in patients with severe sepsis. Therefore, measurement of SgII in the ICU may aid the early identification of patients of high risk, thus improving both the therapy received by these patients and the monitoring of these patients.

TABLE 1
Patient characteristics according to in-hospital survival status

| | Non-survivor | Survivors | P |
|---|---|---|---|
| Variable | n = 46 | n = 159 | value |
| Age | 66 ± 2 | 57 ± 1 | 0.001 |
| Male sex | 26 (57%) | 113 (71%) | 0.07 |
| Diabetes mellitus | 11 (24%) | 31 (20%) | 0.54 |
| Hypertension | 16 (35%) | 58 (37%) | 0.86 |
| Positive blood culture | 12 (26%) | 44 (28%) | 1.00 |
| Ventilator treatment | 40 (87%) | 114 (72%) | 0.04 |
| SAPS II | 50 ± 2 | 41 ± 1 | <0.001 |
| SOFA | 9 ± 1 | 8 ± 1 | 0.02 |
| SgII at baseline (nmol/L) | 0.23 ± 0.02 | 0.18 ± 0.01 | 0.02 |
| hs-cTnT at baseline (μg/L) | 0.536 ± 0.308 | 0.131 ± 0.0.022 | 0.06 |
| SgII at 72 hrs (nmol/L) | 0.19 ± 0.01 | 0.16 ± 0.01 | 0.07 |
| hs-cTnT at 72 hrs (μg/L) | 0.421 ± 0.182 | 0.137 ± 0.025 | 0.08 |

Abbreviations: SAPS II: Simplified Acute Physiology Score II; SOFA: Sequential Organ Failure Assessment score; SgII: secretogranin II; hs-cTnT: highly sensitive assay for cardiac specific Troponin T.

TABLE 2
Univariate and multivariate logistic analysis with in-hospital mortality
as the outcome measure

| | | |
|---|---|---|
| Odds | | |
| ratio | 95% CI. | P value |

Univariate analysis

| | | | |
|---|---|---|---|
| Age, per 1 y increase | 1.04 | 1.02-1.07 | 0.001 |
| Sex (female vs. male) | 1.89 | 0.96-3.72 | 0.07 |
| Diabetes mellitus | 1.30 | 0.59-2.84 | 0.51 |
| Hypertension | 0.92 | 0.46-1.83 | 0.92 |
| SAPS II score, per 1 point increase | 1.04 | 1.02-1.07 | <0.001 |
| SOFA score, per 1 point increase | 1.15 | 1.04-1.28 | 0.01 |
| SgII, per one quartile increase | 1.50 | 1.10-2.05 | 0.01 |
| hs-cTnT, per one quartile increase | 1.35 | 1.00-1.83 | 0.05 |
| Trombocyte count, per 1 unit increase | 1.00 | 1.00-1.00 | 0.71 |
| Creatinine, per 1 log unit increase | 1.56 | 0.93-2.59 | 0.09 |
| Lactate max, per 1 log unit increase | 1.63 | 0.97-2.74 | 0.06 |
| Dobutamine infusion, per 1 unit increase | 1.10 | 0.97-1.26 | 0.14 |
| Dopamine infusion, per 1 unit increase | 1.07 | 0.96-1.21 | 0.23 |
| Norepinephrine infusion, per 1 unit increase | 2.54 | 0.98-6.57 | 0.06 |

Multivariate analysis: final model

| | | | |
|---|---|---|---|
| Age, per 1 y increase | 1.03 | 1.01-1.06 | 0.02 |
| SgII, per one quartile increase | 1.54 | 1.11-2.16 | 0.01 |
| SAPS II score, per 1 point increase | 1.04 | 1.01-1.07 | 0.01 |

Abbreviations:
SAPS II: Simplified Acute Physiology Score II;
SOFA: Sequential Organ Failure Assessment score;
SgII: secretogranin II;
hs-cTnT: highly sensitive assay for cardiac specific Troponin T.

TABLE 3
Univariate and multivariate logistic analysis with ICU mortality as the
outcome measure
Univariate analysis

| | Odds ratio | 95% CI. | p |
|---|---|---|---|
| Age, per 1 y increase | 1.03 | 1.00-1.07 | 0.08 |
| Sex (female vs. male) | 2.58 | 1.00-6.69 | 0.05 |
| Diabetes mellitus | 0.70 | 0.20-2.55 | 0.60 |
| Hypertension | 1.66 | 0.64-4.30 | 0.30 |
| SAPS II score, per 1 point increase | 1.05 | 1.02-1.09 | 0.001 |
| SOFA score, per 1 point increase | 1.18 | 1.02-1.36 | 0.03 |
| SgII, per one quartile increase | 1.70 | 1.07-2.72 | 0.03 |
| hs-cTnT, per one quartile increase | 1.26 | 0.82-1.95 | 0.29 |
| Trombocyte count, per 1 unit increase | 1.00 | 0.99-1.00 | 0.15 |
| Creatinine, per 1 log unit increase | 1.42 | 0.70-2.90 | 0.34 |
| Lactate max, per 1 log unit increase | 1.51 | 0.75-3.01 | 0.25 |
| Dobutamine infusion, per 1 unit increase | 1.09 | 0.92-1.30 | 0.34 |
| Dopamine infusion, per 1 unit increase | 1.15 | 1.01-1.32 | 0.04 |
| Norepinephrine infusion, per 1 unit | 2.64 | 0.87-8.00 | 0.09 |
| increase | | | |

Abbreviations:
SAPS II: Simplified Acute Physiology Score II;
SOFA: Sequential Organ Failure Assessment score;
SgII: secretogranin II;
hs-cTnT: highly sensitive assay for cardiac specific Troponin T.

### EXAMPLE 2

### Prognostic Value of Secretogranin II in Critical Illness: Severe Sepsis

### Introduction

Current risk stratification in patients with sepsis and critical illness is based on multivariable scoring models that have been validated in several independent cohorts. The Simplified Acute Physiology Score (SAPS) II score and the Sequential Organ Failure Assessment (SOFA) score are two strong risk models that incorporate 17 and 6 variables, respectively. Novel risk markers in sepsis should provide incremental information to the established risk models as evaluated by models. The SAPS II and SOFA models integrate information on a wide range of crucial organ systems in sepsis, and to improve risk stratification a new marker should reflect pathophysiology that is not covered by the established models. Additionally, as the established risk models cannot be calculated prior to 24 hrs of hospital admission, a new risk marker that could be readily determined on hospital admission would be of great value for prognosis and selection of therapy in the initial phase of critical illness. Of special importance is identification of patients that will develop circulatory failure (septic shock/medical shock) during the hospitalization as these patients will need targeted therapy with inotropic drugs (dopamine, dobutamine, noreepinephrine, etc) to maintain adequate blood pressure for sustained perfusion of the peripheral organs.

The protein secretogranin II (SgII) is a member of the granin protein family, a family of Ca²⁺-binding proteins with a high proportion of acidic amino acids and multiple dibasic cleavage sites. SgII functions as a pro-hormone, yielding several shorter, functionally active fragments. Currently, there is no information in the literature on SgII in patients with sepsis and critical illness.

### Materials and Methods

### Study Design

This is a substudy of FINNSEPSIS, a multi-center study that comprised 24 intensive care units (ICUs) in Finland (Karlsson S, et al., Intensive Care Med. 2007; 33:435-443). During a 4 month period all adult patients (>18 years) admitted to the participating ICUs were screened daily for the American College of Chest Physicians/Society of Critical Care Medicine criteria of severe sepsis and septic shock (FIG. 3). Patients that fulfilled the criteria of severe sepsis; e.g. (1) known or suspected infection, (2) two or more criteria for systemic inflammatory response syndrome, and (3) at least one new, sepsis-induced organ failure, were included in the FINNSEPSIS study. Patient chart records were used to record a diagnosis of cardiovascular disease (defined as either a history of heart failure, coronary artery disease requiring medication, or former disease-associated procedure), hypertension, and diabetes. Basic laboratory variables recorded during the first 24 hrs after inclusion in the study were peak creatinine and lactate levels, and the lowest platelet count. The maximal doses of vasoactive drugs infused during the first 24 hrs were also recorded. The Simplified Acute Physiology Score (SAPS) II (Le Gall Jr, et al., JAMA. 1993; 270:2957-2963) and the Sequential Organ Failure Assessment (SOFA) score (Vincent J L, et al., Intensive Care Med. 1996; 22:707-710) were calculated 24 hrs after study commencement. In-hospital mortality was recorded and assessed as the primary endpoint, while circulatory failure (septic shock) was considered a secondary endpoint. The study has been conducted according to the Declaration of Helsinki and approved by the local Ethics Committees.

### Biochemical Analysis

The biomarker substudy of FINNSEPSIS included 254 patients for whom a written informed consent for blood sampling could be obtained by the patient or a relative (FIG. 3). The majority of blood samples were collected within 12 hrs of the start of sepsis. For this study, blood samples were available from 238 (94%) of the patients who were included in the biomarker substudy. Blood was drawn from indwelling arterial catheters or by venipuncture, and stored as heparin plasma. Blood samples were centrifuged at room temperature and the serum component aspirated, before the samples were frozen at −20° C. and stored at the individual centers for up to 3 months. Plasma samples were subsequently shipped on dry ice to the Helsinki University Hospital for storage at −80° C. for 4 years pending analysis at the University Hospital in Uppsala, Sweden.

SgII levels from study commencement and 72 hrs thereafter were measured by an in-house made radioimmunoassay with the epitope binding to SgII154-165 (Stridsberg M, et al., Regul Pept. 2008; 148:95-98). N-terminal proB-type natriuretic peptide (NT-proBNP), troponin T (measured with a new, highly sensitive assay (hs-cTnT)) and chromogranin A (CgA) were analyzed by commercially available assays. Body mass index (BMI) was calculated as weight (kg)/height (m)², and creatinine clearance by the Cockcroft-Gault formula.

### Statistical Analysis

Data are presented as median (quartile (Q) 1-3) or absolute numbers and percentages. Categorical data was compared by the Chi-square test or the Fisher Exact test, as appropriate. Spearman rank correlation was used to examine associations between variables. The merit of SgII levels (as quartiles) to predict hospital mortality and septic shock were examined by logistic regression analysis. For hospital mortality the model #1 includes all variables available from the first patient contact (<24 hrs). In this model, all variables significantly associated with hospital mortality in univariate analysis were included in the multivariate model. The merit of analysing SgII levels for prediction of mortality was also examined in model #2 by adjusting for the established risk models in critical illness and sepsis (available after 24 hrs from hospital admission). Variables on study inclusion predictive of septic shock during the hospitalization were assessed by multivariate logistic regression analysis (similar to model #1).

The prognostic accuracy for in-hospital mortality and optimal cut off points were assessed by receiver operating characteristics (ROC) curve analysis with area under the curve (AUCs) presented with 95% confidence intervals (CI). A p value<0.05 was considered statistically significant. Statistical analyses were performed with SPSS for Windows version 14.0 (SPSS, Chicago, Ill.) with the exception of the comparison of ROC AUCs, which was performed with MedCalc for Windows, version 9.5.1.0 (MedCalc Software, Mariakerke, Belgium).

### Results

### Patient Characteristics

There were no major differences concerning clinical characteristics and hospital mortality between patients included in the biomarker substudy cohort and the other patients in FINNSEPSIS. In total 65 patients (27%) died in hospital and 140 (59%) developed septic shock during hospitalization. Characteristics of participating patients according to hospital mortality are presented in Table 4. High levels of cardiac biomarkers on inclusion in the study and after 72 hrs were associated with a poor prognosis as were established risk factors such as increasing age, renal dysfunction, and elevated SAPS II and SOFA score.

### Determinants of SgII Levels in Severe Sepsis

There was a close correlation between SgII levels at baseline and after 72 hrs: 1=0.62, p<0.001. CgA levels on study commencement correlated positively with indices of severity in sepsis such as SAPS II (r=0.36,p<0.001) and SOFA score (1=0.39, p<0.001), as well as to baseline NT-proBNP levels (r=0.35, p<0.001) and NT-proBNP levels after 72 hrs (r=0.30, p<0.001). Furthermore, CgA correlated with the lowest creatinine level measured <24 hrs from study inclusion (r=0.50, p<0.001).

### SgII Levels and Hospital Mortality

SgII levels at baseline and after 72 hrs were higher in hospital non-survivors than in survivors: Admission: 0.21 nmol/L (0.15-0.28 nmol/L) vs. 0.16 nmol/L (0.13-0.22 nmol/L), p<0.001, and after 72 hrs: 0.17 nmol/L (0.14-0.24 nmol/L) vs. 0.15 nmol/L (0.12-0.18 nmol/L), p=0.003. SgII levels on admission differentiated better between patients with a favorable and poor prognosis than the corresponding hs-cTnT and NT-proBNP levels (FIG. 4). SgII measured on inclusion in the study was also the strongest predictor for hospital mortality of all variables available in the first 24 hrs in sepsis (Table 5). Furthermore, a SgII level>0.17 nmol/L measured during the first hours of severe sepsis provided incremental prognostic information to the established risk models SOFA and SAPS II score that were calculated after 24 hrs (Table 6).

The prognostic accuracy of SgII early after hospital admission for critical illness and severe sepsis was superior to what was found for CgA (FIG. 5). In contrast to SgII, CgA levels measured within 24 hrs of the start of severe sepsis were not an independent predictor for hospital mortality, including in models in which SgII was excluded from the analysis.

It has also been shown that SgII is a powerful marker for predicting whether ICU patients will survive to ICU discharge (FIG. 7).

### SgII Levels and Septic Shock

SgII levels on admission were higher in patients who developed septic shock during the hospitalization than in those who did not develop septic shock: 0.18 nmol/L (0.15-0.24 nmol/L) vs. 0.15 nmol/L (0.12-0.21 nmol/L), p=0.004, and was a stronger predictor of patients developing septic shock than levels of hs-cTnT and NT-proBNP measured on inclusion in the study (FIG. 6). SgII levels were also independently associated with septic shock after adjusting for all other variables available on hospital admission, including established cardiac biomarkers and CgA (Table 7).

### CONCLUSION

The principal finding of this study is the strong prognostic information obtained by measuring SgII levels on hospital admission in patients with sepsis and critical illness. SgII is superior to the state-of-the-art biomarkers NT-proBNP and hs-cTnT, and provides incremental information to that obtained by calculating SOFA and SAPS II score after 24 hrs in hospital. In this cohort, a quartile increase in admission SgII levels equated to a 60% increase in risk (odds) for hospital mortality after adjusting for all other variables on study inclusion, and a SgII level >0.17 nmol/L indicated a 110% and 95% increase in odds of death after adjusting for SOFA and SAPS II score, respectively. Similarly, a quartile increase in admission SgII levels indicated 60% increase in odds for circulatory failure during the hospitalization after adjustment for other variables on study inclusion, including CgA levels.

TABLE 4
Characteristics of the study patients according to in-hospital survival status

| | Non-survivors | | Survivors | | p |
|---|---|---|---|---|---|
| Variable | (n = 65) | | (n = 169) | | value |
| Age | 66 | (57-76) | 59 | (48-70) | <0.001 |
| Male sex | 39 | (60%) | 122 | (71%) | 0.12 |
| Body mass index | 26 | (23-28) | 26 | (23-29) | 0.58 |
| Cardiovascular disease | 27 | (42%) | 38 | (22%) | 0.003 |
| Diabetes | 24 | (41%) | 34 | (22%) | 0.005 |
| Hypertension | 24 | (44%) | 68 | (41%) | 0.73 |
| Peak creatinine levels <24 h (μmol/L) | 129 | (75-267) | 98 | (70-159) | 0.021 |
| Lowest creatinine clearance <24 hrs | 45 | (22-90) | 70 | (44-110) | 0.001 |
| (mg/min) | | | | | |
| Peak lactate levels <24 hrs (mmol/L) | 3.0 | (1.7-4.5) | 1.9 | (1.1-3.0) | <0.001 |
| Lowest platelet count <24 hrs (×10⁹/L) | 126 | (79-224) | 148 | (88-272) | 0.16 |
| SAPS II score | 54 | (41-64) | 40 | (30-50) | <0.001 |
| SOFA score | 10 | (7-13) | 8 | (6-10) | <0.001 |
| SgII on inclusion (nmol/L) | 0.21 | (0.15-0.28) | 0.16 | (0.13-0.22) | <0.001 |
| CgA on inclusion (nmol/L) | 14.0 | (7.4-27.4) | 9.1 | (5.9-15.8) | 0.002 |
| NT-proBNP on inclusion (pg/mL) | 7454 | (2605-20378) | 3460 | (1063-9470) | 0.004 |
| hs-cTnT on inclusion (μg/L) | 0.054 | (0.022-0.227) | 0.035 | (0.015-0.113) | 0.047 |
| SgII at 72 hrs (nmol/L) | 0.17 | (0.14-0.24) | 0.15 | (0.12-0.18) | 0.003 |
| CgA at 72 hrs (nmol/L) | 16.2 | (9.0-31.1) | 9.8 | (6.0-18.0) | 0.001 |
| NT-proBNP at 72 hrs (pg/mL) | 5516 | (1364-17241) | 2191 | (606-6038) | 0.002 |
| hs-cTnT at 72 hrs (μg/L) | 0.054 | (0.019-0.224) | 0.034 | (0.015-0.088) | 0.047 |

Abbreviations:
SAPS II: Simplified Acute Physiology Score II;
SOFA: Sequential Organ Failure Assessment score;
SgII, secretogranin II;
CgA: chromogranin A;
NT-proBNP: N-terminal pro-B type natriuretic peptide; and
hs-cTnT, troponin T measured by a highly sensitive assay.

TABLE 5
Predictors for hospital mortality in sepsis that available on hospital
admission

| | | |
|---|---|---|
| Odds ratio | 95% CI. | p |

Univariate analysis

| | | | |
|---|---|---|---|
| Age, per 1 y increase | 1.05 | 1.02-1.07 | 0.001 |
| Gender (female vs. male) | 1.80 | 0.92-3.53 | 0.09 |
| Body mass index (per 1 unit increase) | 0.99 | 0.93-1.05 | 0.63 |
| Cardiovascular disease | 2.54 | 1.37-4.68 | 0.003 |
| Diabetes | 2.56 | 1.35-4.87 | 0.004 |
| Hypertension | 1.12 | 0.60-2.07 | 0.73 |
| Creatinine clearance, per 1 unit | 0.45 | 0.29-0.71 | <0.001 |
| increase | | | |
| Lactate, per 1 unit increase | 2.25 | 1.43-3.54 | <0.001 |
| Platelet count, per 1 unit increase | 0.82 | 0.60-1.13 | 0.23 |
| hs-cTnT on inclusion, per 1 unit | 1.28 | 1.03-1.60 | 0.025 |
| increase | | | |
| NT-proBNP on inclusion, per 1 unit | 1.32 | 1.08-1.61 | 0.008 |
| increase | | | |
| CgA on inclusion, per quartile increase | 1.52 | 1.16-1.99 | 0.002 |
| CgB on inclusion, per quartile increase | 1.47 | 1.13-1.93 | 0.005 |
| SgII on inclusion, per quartile increase | 1.67 | 1.26-2.20 | <0.001 |

Multivariate analysis: final model

| | | | |
|---|---|---|---|
| SgII on inclusion, per quartile | 1.59 | 1.05-2.40 | 0.029 |
| increase | | | |
| Age | 1.04 | 1.003-1.07 | 0.030 |

Abbreviations:
hs-cTnT: highly sensitive assay for cardiac troponin T;
NT-proBNP: N-terminal B-type natriuretic peptide;
CgA: chromogranin A;
CgB: chromogranin B; and
SgII: secretogranin II.

TABLE 6
Predictors for hospital mortality in sepsis that are available after 24 hrs

| | | |
|---|---|---|
| Odds ratio | 95% CI. | p |

Model 1: SgII and SOFA score

| | | | |
|---|---|---|---|
| SOFA score | 3.22 | 1.42-7.30 | 0.005 |
| SgII >0.17 nmol/L | 2.16 | 1.13-4.12 | 0.020 |

Model 2: SgII and SAPS II score

| | | | |
|---|---|---|---|
| SAPS II score | 1.05 | 1.03-1.07 | <0.001 |
| SgII >0.17 nmol/L | 1.95 | 1.02-3.74 | 0.043 |

Abbreviations:
SOFA; Sequential Organ Failure Assessment score;
SgII: secretogranin II; and
SAPS II: Simplified Acute Physiology Score II.
One unit increase for SOFA score correspond to an increase in the natural logarithm of SOFA score, while SgII was divided by the optimal cutoff 0.17 nmol/L for prediction of hospital mortality.

TABLE 7
Association between variables available in the early phase of sepsis and
circulatory failure (shock) during the hospitalization

| | | |
|---|---|---|
| Odds ratio | 95% CI. | P value |

Univariate analysis

| | | | |
|---|---|---|---|
| Age, per 1 y increase | 1.02 | 1.01-1.04 | 0.007 |
| Gender (female vs. male) | 0.93 | 0.56-1.70 | 0.93 |
| Body mass index, per 1 unit increase | 1.04 | 0.98-1.10 | 0.21 |
| Cardiovascular disease | 1.67 | 0.92-3.05 | 0.09 |
| Diabetes | 1.05 | 0.57-1.93 | 0.87 |
| Hypertension | 1.09 | 0.63-1.87 | 0.76 |
| Creatinine clearance, per 1 unit | 0.76 | 0.51-1.13 | 0.18 |
| increase | | | |
| Lactate, per 1 unit increase | 1.90 | 1.24-2.90 | 0.003 |
| Platelet count, per 1 unit increase | 0.87 | 0.64-1.17 | 0.34 |
| hs-cTnT on inclusion, per 1 unit | 1.26 | 1.03-1.53 | 0.024 |
| increase | | | |
| NT-proBNP on inclusion, per 1 unit | 1.21 | 1.02-1.44 | 0.029 |
| increase | | | |
| CgA on inclusion, per quartile | 1.37 | 1.08-1.74 | 0.009 |
| increase | | | |
| SgII on inclusion, per quartile | 1.49 | 1.17-1.91 | 0.002 |
| increase | | | |

Multivariate analysis: final model

| | | | |
|---|---|---|---|
| SgII on inclusion, per quartile | 1.58 | 1.16-2.15 | 0.004 |
| increase | | | |

Abbreviations:
hs-cTnT: highly sensitive assay for cardiac Troponin T;
NT-proBNP: N-terminal B-type natriuretic peptide;
CgA: chromogranin A; and
SgII: secretogranin II.
Due to logarithmic transformation, one unit increase for creatinine clearance, platelet count, lactate, hs-cTnT, and NT-proBNP levels correspond to one unit increase in the natural logarithm of these variables.

### EXAMPLE 3

### Circulating Secretogranin II Levels in Non-Septic Patients with Critical Illness: Results from ICU Patients Requiring Mechanical Ventilation

### Introduction

Mechanically ventilated intensive care unit (ICU) patients are critical ill and have a high short-term mortality rate. Secretogranin II (SgII) is a strong risk marker in critical illness and severe sepsis, and to supplement these data we have now examined SgII levels in mechanical ventilated non-septic patients with critical illness.

### Methods

### Study Design

For this study we included 32 non-septic, non-cardiac adult patients (18 years or older) with a long-term requirement (>48 hrs) of mechanical ventilation admitted to the ICU of Akershus University Hospital, Lorenskog, Norway. Akershus University Hospital is a secondary referral and teaching hospital in metropolitan Oslo with a catchment area of approximately 320 000 people. The ICU is a combined medical and surgical ICU. All surgical specialties except neuro-, cardiac- and transplant surgery are covered in the hospital. The primary end-point was short-term mortality (≧30 days mortality), while long-term mortality (>30 days mortality) was the secondary end-point.

The research was carried out in accordance with the Declaration of Helsinki (2000) of the World Medical Association and the study protocol was approved by the Regional Ethics Committee before the initiation of the study.

### Analysis of SgII

Blood samples were drawn from an antecubital vein 48 hours after ICU admission. Serum was frozen and later stored at −80° C. until analysis. SgII levels were measured by an in-house made radioimmunoassay with the epitope binding to SgII154-165.

### Statistical Analysis

Data are presented as median (quartile 1-3). SgII levels were compared by the Mann-Whitney U-test. Long-term prognosis by SgII quartiles were compared by Kaplan-Meier plot and assessed by the Log rank test. Statistical analyses were performed with SPSS for Windows version 14.0 (SPSS, Chicago, Ill.).

### Results

### Patients

Thirteen patients were admitted due to acute respiratory failure, 8 due to gastrointestinal disease, 3 due to trauma, and 9 patients due to various other causes (intoxication, complications due to surgery and neurological conditions). None of the included patients were suffering from sepsis.

Secretogranin II and Prognosis

The 30 days mortality rate (short term mortality rate) in this cohort was 34% (11 out of 32 patients). SgII levels were higher in the patients dying before 30 days than the day-survivors: 0.16 (0.11-0.21) vs. 0.11 (0.05-0.15), p=0.08 (FIG. 8). Similarly, the patients with elevated SgII levels early after ICU admission also had a poor long term prognosis (p=0.02 for difference across SgII quartiles, FIG. 9).

### Discussion

The main results of this study in a heterogeneous group of non-septic patients with critical illness is that SgII levels measured early after hospitalization can be used to assess short-term and long-term prognosis. SgII is thus a powerful new biomarker in critical illness that is not restricted to a particular underlying condition.

1. A method of determining the prognosis of a condition which requires critical care, said method comprising determining the level of SgII, or fragments thereof, in a subject.

2. A method of determining whether an individual is suffering from a condition which requires critical care, said method comprising determining the level of SgII, or fragments thereof, in a subject.

3. A method of determining the severity of a condition which requires critical care, said method comprising determining the level of SgII, or fragments thereof, in a subject.

4. The method of claim 1, wherein said condition which requires critical care is a condition requiring admittance to an intensive care unit or trauma centre.

5. The method of claim 1, wherein an increased level of SgII, or fragments thereof, in said subject is indicative of a condition which requires critical care.

6. The method of claim 1, wherein the level of SgII, or fragments thereof, in said subject is compared to a control level.

7. The method of claim 1, wherein serial determinations of the level of SgII, or fragments thereof, are made.

8. The method of claim 1, wherein said condition is selected from the group consisting of sepsis, severe sepsis, septic shock, acute respiratory failure, gastrointestinal disease, trauma, any disease or condition which requires mechanical ventilation, or complications from surgery.

9. The method of claim 8, wherein said condition is selected from the group consisting of sepsis, severe sepsis or septic shock.

10. The method of claim 1, wherein a circulating level of SgII of 0.20 nmol/L determines the presence of a condition which requires critical care, determines that a condition which requires critical care is severe, or determines that a patient has a poor prognosis.

11. The method of claim 1, wherein the SgII fragment is secretoneurin (SN).

12. The method of claim 1, wherein said level of SgII, or fragments thereof, is measured in a body fluid or tissue sample from said subject.

13. The method of claim 12, wherein said body fluid is a circulatory sample.

14. The method of claim 12, wherein said body fluid is blood, serum, plasma, saliva or urine.

15. The method of claim 1, wherein said method is used to determine the severity of said condition in said subject.

16. The method of claim 2, wherein said method is used to determine the prognosis of said condition in said subject.

17. The method of claim 1, wherein said method is used to diagnose activated pathophysiological pathways in a subject suffering from a condition which requires critical care, to identify a subject requiring more intensive monitoring, or to identify a subject which might benefit from early therapeutic intervention.

18. The method of claim 1, wherein said method is used to monitor the progress of said condition, to assess the effectiveness of therapy for said condition, or to monitor the progress of said condition.

19. The method of claim 1, wherein said determination is carried out within 24 or 48 hours of said subject having contact with a medical professional or being admitted to a critical care setting.

20. The method according to claim 1, wherein said methods are used in conjunction with other known methods for determining severity or determining prognosis of a condition which requires critical care.

21. The method of claim 1, wherein the wherein the level of SgII is determined by radioimmunoassay or Enzyme-Linked ImmunoSorbent Assay

22. The method of claim 21, wherein the radioimmunoassay measures the epitope SgII 154-165.

23. A method of imaging of a subject which has a condition which requires critical care or which potentially has a condition which requires critical care, comprising the administration of an appropriate amount of a binding entity which can target SgII, or fragments thereof, to the subject and detecting the presence and/or amount and/or the location of the binding entity in the subject.

24. A kit for diagnosing or for determining severity or prognosis of a critical care condition using a method as defined in claim 1, which comprises an agent suitable for determining the level of SgII, or fragments thereof, in a sample.

25. A method of testing the therapeutic potential of a substance for the treatment of critical illness, comprising administering a test substance to an experimental animal suffering from a critical illness and determining the level of SgII, or fragments thereof, in said animal.


SEQUENCE LISTING (1 sequence)

SEQ ID NO: 1 - protein, 589 aa, Homo sapiens
SFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM